(19) United States
(12) Patent Application Publication
(10) Pub. No.: US 2026/0144514 A1
(43) Pub. Date: May 28, 2026

(54) SYSTEMS AND METHODS FOR MONITORING HEALTH DATA

(71) Applicant: Esperto Medical, Inc., Irvine, CA (US)

(72) Inventors: Alaina Brinley RAJAGOPAL, Irvine, CA (US); Aditya RAJAGOPAL, Irvine, CA (US); Raymond JIMENEZ, Gardena, CA (US); Vernon Norviel, Evergreen, CO (US)

(21) Appl. No.: 19/451,619

(22) Filed: Jan. 16, 2026

Related U.S. Application Data
(63) Continuation-in-part of application No. PCT/US2024/038624, filed on Jul. 18, 2024.
(60) Provisional application No. 63/527,553, filed on Jul. 18, 2023.
(60) Provisional application No. 63/527,549, filed on Jul. 18, 2023.
(60) Provisional application No. 63/527,543, filed on Jul. 18, 2023.
(60) Provisional application No. 63/527,552, filed on Jul. 18, 2023.

(51) Int. Cl. A61B 8/04; A61B 8/00; A61B 8/13; G16H 10/60

(52) U.S. Cl. CPC A61B 8/04; A61B 8/13; A61B 8/4281; A61B 8/4488; G16H 10/60

(57) ABSTRACT

In some implementations, the device may include a plurality of ultrasound sensors configured to capture tomographical information of a physiological structure. In addition, the device may include an ultrasound coupling medium on each of the plurality of ultrasound sensors. The device may include a processing device configured to: apply beamforming techniques to the ultrasound sensors; capture, using the plurality of ultrasound sensors, bodily data of overlapping volumes of the physiological structure; process the bodily data to generate a vessel location model; and locate a blood vessel from the generated vessel location model.

### CROSS-REFERENCE TO RELATED APPLICATION

This application is a continuation-in-part of International Patent Application No. PCT/US2024/038624, filed Jul. 18, 2024, which claims the benefit of and priority to U.S. Provisional Patent Application No. 63/527,553, filed on Jul. 18, 2023, U.S. Provisional Application No. 63/527,552, filed on Jul. 18, 2023, U.S. Provisional Application No. 63/527,549, filed on Jul. 18, 2023, and U.S. Provisional Application No. 63/527,543, filed on Jul. 18, 2023, each of which is entirely incorporated herein by reference.

### BACKGROUND

Blood pressure is an essential vital sign routinely used to manage patient care. Methods of blood pressure measurement are typically cumbersome and bulky. Typical methods, such as using stethoscopes in conjunction with a sphygmomanometer and blood pressure arm/wrist cuff(s), have several limitations, including susceptibility to ambient noise, patient discomfort, and inability to obtain a continuous blood pressure measurement. An alternative is invasive blood pressure measurement, such as using arterial catheters. While this provides much higher quality data than an external cuff its invasive nature also produces much higher risks, including infection, hemorrhage, or ischemia. Alternative non-invasive modalities for measuring blood pressure are highly desirable, particularly as hypertension has become an increasingly prevalent medical issue in both the United States and the rest of the world.

### SUMMARY

Systems and methods described herein are directed to a blood vessel locating device.

In one embodiment, a blood vessel locating device comprises: a plurality of ultrasound sensors configured to capture tomographical information of a physiological structure; an ultrasound coupling medium disposed on an ultrasound sensor of the plurality of ultrasound sensors; and a processing device configured to: apply beamforming techniques to the ultrasound sensors; capture, using the plurality of ultrasound sensors, bodily data of overlapping volumes of the physiological structure; process the bodily data to generate a vessel location model; and locate a blood vessel from the generated vessel location model.

In some embodiments, the ultrasound coupling medium is disposed on each ultrasound sensor of the plurality of ultrasound sensors.

In some embodiments, the ultrasound sensor comprises a transducer. In some embodiments, each of the plurality of ultrasound sensors of the blood vessel locating device comprises a transducer.

In some embodiments, the transducer comprises low pixel-count arrays. In some embodiments, each of the transducers of the plurality of ultrasound sensors comprises low pixel-count arrays.

In some embodiments, each of the transducers uses phased-array technology.

In some embodiments, the ultrasound coupling medium of the blood vessel locating device comprises silicone.

In some embodiments, the ultrasound coupling medium of the blood vessel locating device comprises hydrogel.

In some embodiments, the ultrasound coupling medium of the blood vessel locating device comprises gel.

In some embodiments, the processing device of the blood vessel locating device captures bodily data of overlapping volumes of the physiological structure by: acquiring velocity of blood moving through the blood vessel; acquiring resonance response of the blood vessel to an audio-frequency sound stimulus; and capturing image data of the blood vessel and surrounding structures, wherein the surrounding structures include but are not limited to organs, tissues, bones, muscles, tendons and other internal components of the physiological structure near the blood vessel that are detectable by ultrasound.

In some embodiments, the ultrasound sensor of the plurality of ultrasound sensors is configured to apply the audio-frequency sound stimulus to the blood vessel.

In some embodiments, the processing device of the blood vessel locating device is further configured to: compare the velocity of the blood vessel to a first threshold and the resonance response of the blood vessel to a second threshold; and track the blood vessel if the acquired velocity of the blood vessel is greater than a first threshold and the acquired resonance response is greater than a second threshold.

In some embodiments, the processing device of the blood vessel locating device processes the bodily data to generate the vessel location model by: generating the vessel location model using the captured bodily data; and acquiring, using the bodily data, arterial stiffness, wall thickness, and arterial diameter of the blood vessel to apply to the vessel location model.

In some embodiments, the processing device of the blood vessel locating device is further configured to: generate an image of the blood vessel, the surrounding structures, pathologies, and/or properties of the physiological structure based on the captured image data.

In some embodiments, the blood vessel locating device further comprises a display, wherein the display is configured to display the bodily data.

In some embodiments, blood vessel locating device further comprises a display, wherein the display is configured to display the generated image of the blood vessel, surrounding structures, pathologies, and/or properties.

In some embodiments, the processing device is further configured to: (i) determine a plurality of blood pressure measurements of a subject from the blood vessel located in the physiological structure and (ii) using the plurality of blood pressure measurements and medical records of the subject, determine a health status of the subject.

In some embodiments, the processing device is further configured to: (I) generate an alert threshold based at least in part on the health status of the subject and (II) provide an alert to the subject or an external party upon determining blood pressure measurements of the plurality of blood pressure measurements are greater than or less than the alert threshold.

In some embodiments, the processing device is further configured to (I) monitor a variability in the plurality of blood pressure measurements over time and (II) provide the variability in the plurality of blood pressure measurements over time to electronic medical records of the subject.

In some embodiments, the processing device is further configured to (I) determine a stress level of the subject from the plurality of blood pressure measurements and (II) provide an alert to the subject or an external party upon the stress level of the subject reaching a threshold stress level.

In one embodiment, a method comprises: applying beamforming techniques to a plurality of ultrasound sensors, wherein an ultrasound sensor of the plurality of ultrasound sensors comprises an ultrasound coupling medium disposed thereon; capturing, using the plurality of ultrasound sensors, bodily data of overlapping volumes of a physiological structure; processing the bodily data to generate an vessel location model; and locating a blood vessel from the generated vessel location model.

In some embodiments, each of the plurality of ultrasound sensors comprises the ultrasound coupling medium disposed thereon.

In some embodiments, the ultrasound sensor or the plurality of ultrasound sensors are coated with the ultrasound coupling medium.

In some embodiments, each of the plurality of ultrasound sensors comprises a transducer.

In some embodiments, each of the transducers of the plurality of ultrasound sensors comprises low pixel-count arrays.

In some embodiments, each of the transducers uses phased-array technology.

In some embodiments, the ultrasound coupling medium comprises silicone.

In some embodiments, the ultrasound coupling medium comprises hydrogel.

In some embodiments, the ultrasound coupling medium comprises gel.

In some embodiments, capturing bodily data of overlapping volumes of the physiological structure comprises: acquiring velocity of blood moving through the blood vessel; acquiring resonance response of the blood vessel by applying audio-frequency sound stimulus using the plurality of ultrasound sensors; and capturing image data of the blood vessel and surrounding structures, wherein the surrounding structures comprise organs, tissues, bones, muscles, tendons, and other internal components of the physiological structure near the blood vessel that are detectable by ultrasound.

In some embodiments, the method further comprises (i) determining a plurality of blood pressure measurements of a subject from the blood vessel located in the physiological structure and (ii) from the plurality of blood pressure measurements and medical records of the subject, determining a health status of the subject.

In some embodiments, the method further comprises (I) generating an alert threshold based at least in part on the medical records of the subject and (II) providing an alert to the subject or an external party upon determining blood pressure measurements of the plurality of blood pressure measurements are greater than or less than the alert threshold.

In some embodiments, the method further comprises (I) monitoring a variability in the plurality of blood pressure measurements over time and (II) providing the variability in the plurality of blood pressure measurements over time to electronic medical records of the subject.

In some embodiments, the method further comprises (I) determining a stress level of the subject from the plurality of blood pressure measurements and (II) providing an alert to the subject or an external party upon the stress level of the subject reaching a threshold stress level.

In some embodiments, a blood vessel monitoring device may include a plurality of non-receiving transducers configured to transmit energy towards a blood vessel. A non-receiving transducer or each non-receiving transducer may be connected to a coupling medium. The device may further include a processing device configured to direct energy at a first frequency towards the blood vessel, capture a first set of data corresponding to a first impedance measurement of the non-receiving transducer of the plurality of non-receiving transducers, direct energy at a second frequency towards the blood vessel, capture a second set of data corresponding to a second impedance measurement of the non-receiving transducer of the plurality of non-receiving transducers, and compare the first and second sets of data.

In some embodiments, each of the plurality of non-receiving transducers may be non-receiving acoustic transducers.

In some embodiments, each of the plurality of non-receiving transducers may be non-receiving ultrasound transducers.

In some embodiments, the coupling medium may be an ultrasound coupling medium.

In some embodiments, the first frequency and the second frequency are different.

In some embodiments, additional frequencies may be applied towards the blood vessel until a lowest impedance is found by comparing nth sets of data to nth+1 sets of data, where the lowest impedance may be indicative of a resonant frequency of the blood vessel wall.

In some embodiments, the processing device is further configured to: (i) determine a plurality of blood pressure measurements of a subject from the attribute data of the blood vessel and (ii) using the plurality of blood pressure measurements and medical records of the subject, determine a health status of the subject.

In some embodiments, the processing device is further configured to: (I) generate an alert threshold based at least in part on the health status of the subject and (II) provide an alert to the subject or an external party upon determining blood pressure measurements of the plurality of blood pressure measurements are greater than or less than the alert threshold.

In some embodiments, the processing device is further configured to (I) monitor a variability in the plurality of blood pressure measurements over time and (II) provide the variability in the plurality of blood pressure measurements over time to an electronic medical record of the subject.

In some embodiments, the processing device is further configured to (I) determine a stress level of the subject from the plurality of blood pressure measurements and (II) provide an alert to the subject or an external party upon the stress level of the subject reaching a threshold stress level.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates an example computing environment of a measuring device in accordance with some embodiments.

FIG. 2 illustrates an example ultrasound sensor or transducer that may be used in the measuring device in accordance with some embodiments.

FIG. 3 illustrates an example computing environment of a measuring device comprising one or more components in accordance with some embodiments.

FIG. 4 illustrates an example process of a measuring device in accordance with some embodiments.

FIG. 5 illustrates an example image of an internal part of a physiological structure that is being monitored by the measuring device in accordance with some embodiments.

FIG. 6 illustrates an example image of a physiological structure with various internal parts of the human body that may be identified and monitored using the measuring device in accordance with some embodiments.

FIG. 7 illustrates example images of an internal part of a physiological structure that are generated by the measuring device in accordance with some embodiments.

FIG. 8 illustrates an example chip set that can be utilized in implementing architectures and methods in accordance with various implementations of the disclosure.

FIG. 9 illustrates an example methodology for generating a blood pressure measurement as a function of a determined resonant frequency of vibration of a wall of a blood vessel, in accordance with various embodiments of the presently disclosed technology.

FIG. 10 is a companion figure to FIG. 9 that depicts how the methodology of FIG. 9 may be implemented using a computing component, in accordance with various embodiments of the presently disclosed technology.

FIG. 11 illustrates another example methodology for generating a blood pressure measurement as a function of a determined resonant frequency of vibration of a wall of a blood vessel, in accordance with various embodiments of the presently disclosed technology.

FIG. 12 is a companion figure to FIG. 11 that depicts how the methodology of FIG. 11 may be implemented using a computing component, in accordance with various embodiments of the presently disclosed technology.

FIG. 13 illustrates another example methodology for generating a blood pressure measurement as a function of a determined resonant frequency of vibration of a wall of a blood vessel, in accordance with various embodiments of the presently disclosed technology.

FIG. 14 is a companion figure to FIG. 13 that depicts how the methodology of FIG. 13 may be implemented using a computing component, in accordance with various embodiments of the presently disclosed technology.

FIG. 15 illustrates an example chip set that can be utilized in implementing architectures and methods in accordance with various implementations of the disclosure.

FIG. 16 illustrates an example workflow for determining generating a health status of a subject.

FIG. 17 illustrates an example workflow for determining a personalize target blood pressure range for a subject.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

The following description provides specific details for an understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of′ or “consisting essentially of” the recited components.

In one example of vital sign detection and monitoring, a measuring device containing a plurality of ultrasound sensors and a processing device may be used to detect vital signs and internal parts of a physiological structure. Internal parts of a physiological structure may include blood vessels, organs, tissues, bones, muscles, tendons, etc. Internal parts of the physiological structure may also include various pathologies including, but not limited to, a fracture, an abscess, a tumor, cellulitis, stones, etc. The measuring device may further use the low or high frequency sound waves to determine properties of the blood vessel and other internal parts of the physiological structure.

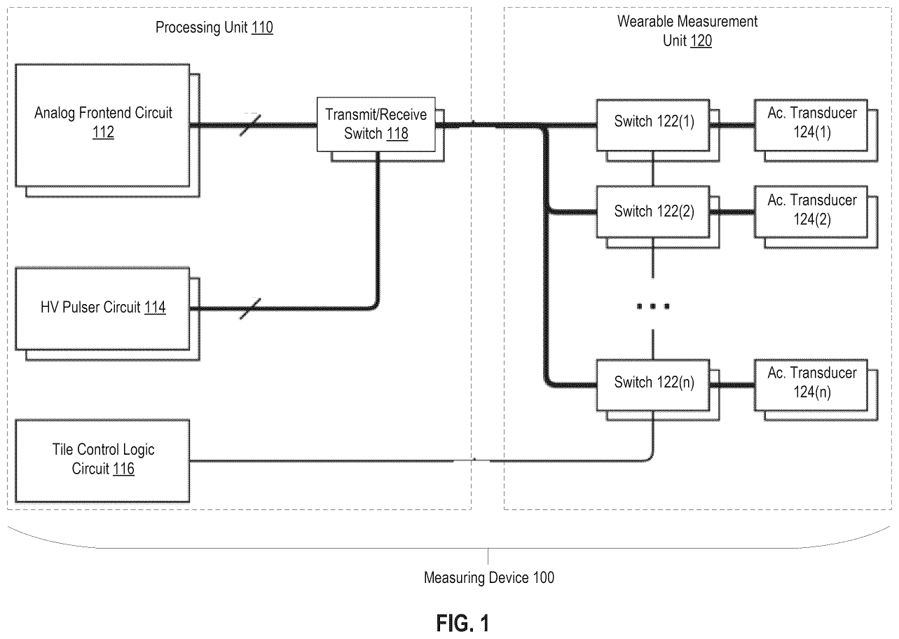

FIG. 1 illustrates an example computing environment of a measuring device 100 that may be attached to a location of a physiological structure to detect and monitor vital signs and internal parts of the physiological structure. Measuring device 100 may include a processing unit 110 and a wearable measurement unit 120 (e.g., an adhesive patch, a wearable cuff such as an armband or wristband, etc.). As depicted, the wearable measurement unit may comprise a plurality of acoustic transducers (i.e., acoustic transducers 124(1)-(n)). In certain embodiments, the acoustic transducers 124(1)-(n) may be acoustic transmitters that do not include a receiving function (e.g., a microphone or similar acoustic receiving feature [also described throughout as one form of non-receiving transducer]). In certain embodiments, measuring device 100 may comprise a single/unitary physical device comprising processing unit 110 and wearable measurement unit 120. In other embodiments processing unit 110 and wearable measurement unit 120 may be separate physical devices in operative communication with each other.

As depicted, processing unit 110 may comprise an analog frontend circuit 112, a high voltage (HV) pulser circuit 114, a tile control logic circuit 116, and a transmit/receive switch 118. HV pulser circuit 114 can generate audio signals (e.g., high frequency sound waves) of different waveforms and frequencies. Analog frontend circuit 112 can amplify the audio signals generated by HV pulser circuit 114. Tile control logic circuit 116 can work in combination with transmit/receive switch 118 and switches 122(1)-(n) (of wearable measurement device 120) to effectively multiplex the audio signals generated and amplified by analog frontend circuit 112, a high voltage (HV) pulser circuit 114 respectively.

As depicted, wearable measuring unit 120 comprises switches 122(1)-(n) and acoustic transducers 121(1)-(n). In certain embodiments, each of acoustic transducers 124(1)-(n) may comprise low pixel-count arrays for transmitting and/or receiving acoustic energy.

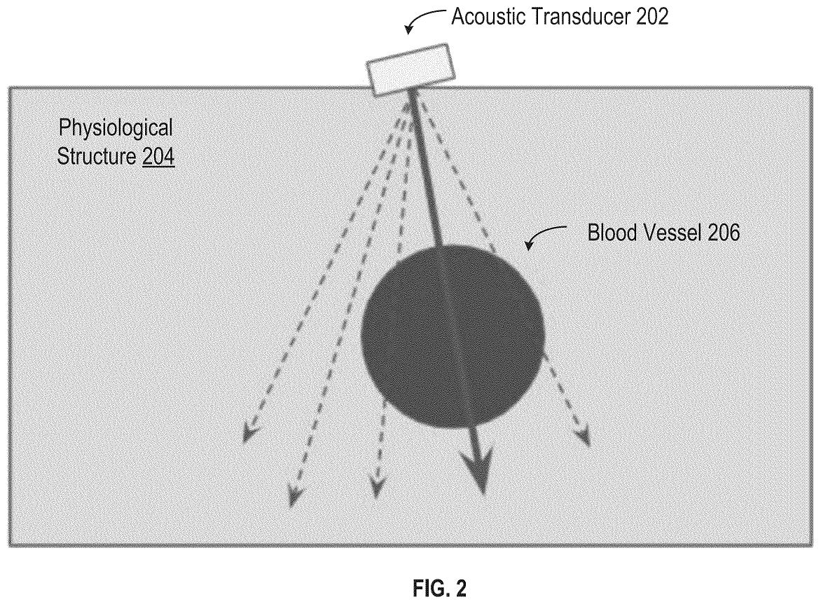

FIG. 2 illustrates an example ultrasound sensor or transducer 202 that may be used in the measuring device to transmit high frequency sound waves. As depicted, acoustic transducer 202 can transmit audio energy (sometimes referred to herein as acoustic energy) at/towards a blood vessel 206 through a physiological structure 204 (e.g., human tissue). Generally, a plurality of ultrasound sensors are configured to capture tomographical information of a physiological structure. An ultrasound sensor may be an ultrasound transducer. An ultrasound sensor may be used to detect ultrasound energy by transmitting and receiving high frequency sound waves. The high frequency sound waves may be at different frequencies and produce different pitches and sounds according to the frequency. Different frequencies of high frequency sound waves may help obtain data on different vital signs and internal parts of a physiological structure. The measuring device may generate and detect various frequencies of high frequency sound waves to obtain some and/or all of the vital signs and internal parts of the physiological structure.

In certain embodiments, an ultrasound sensor may be an ultrasound and/or acoustic transmitter (e.g., a non-receiving transducer). The transmitter may be positioned in a similar manner to the ultrasound transducer, however, the transmitter may only transmit acoustic or ultrasound energy towards the physiological structure without receiving acoustic energy in reflection from the physiological structure. In another embodiment, the transmitter may be an ultrasound transducer with a “transmit only” mode. In such embodiments that include a transmitter, the impedance detected from the transmitter may be used to detect the resonance of the blood vessel, which may be used to obtain vital signs and internal parts of the physiological structure.

Using the ultrasound sensors, either in combination or separately from other components, including low pixel-count arrays, such as 32 elements per ultrasound sensor, phased-array technology and beamforming techniques, the measuring device may collect data of overlapping volumes of a physiological structure. The measuring device may use the collected data to determine measurements associated with vital signs and internal parts of the physiological structure. A vital sign may include blood pressure, heart rate, temperature, respiratory rate, oxygen saturation, as well as other vital signs measurable from the extremities or body of a patient. An internal part of the physiological structure may include a blood vessel, artery, vein, organ (i.e., heart, lungs, liver, kidneys, small intestine, large intestine, stomach, brain, etc.), bone, tissue, muscle, tendon, etc., and the internal part may be within the overlapping volumes of the physiological structure. An internal part of the physiological structure may also include various pathologies including, but not limited to, a fracture, an abscess, a tumor, cellulitis, stones, etc. Measurements of a blood vessel may include the blood vessel's dimensions, i.e., blood vessel stiffness, blood vessel wall thickness, vessel radius, arterial diameter, intima-media thickness, etc., and attributes of the blood vessel, including resonance response of the blood vessel, the velocity of blood flow in the blood vessel, distance between the blood vessel and the measuring device, and plaque thickness in the blood vessel. The measuring device may apply LaPlace's Law or the LaPlace Theorem to obtain measurements of a blood vessel. The measuring device may also use the collected data to produce images of a blood vessel and/or other internal parts within the overlapping volumes of the physiological structure. The measuring device may also use collected impedance data (e.g., from a transmitter) to produce location data of a blood vessel and/or other internal parts within the overlapping volumes of the physiological structure.

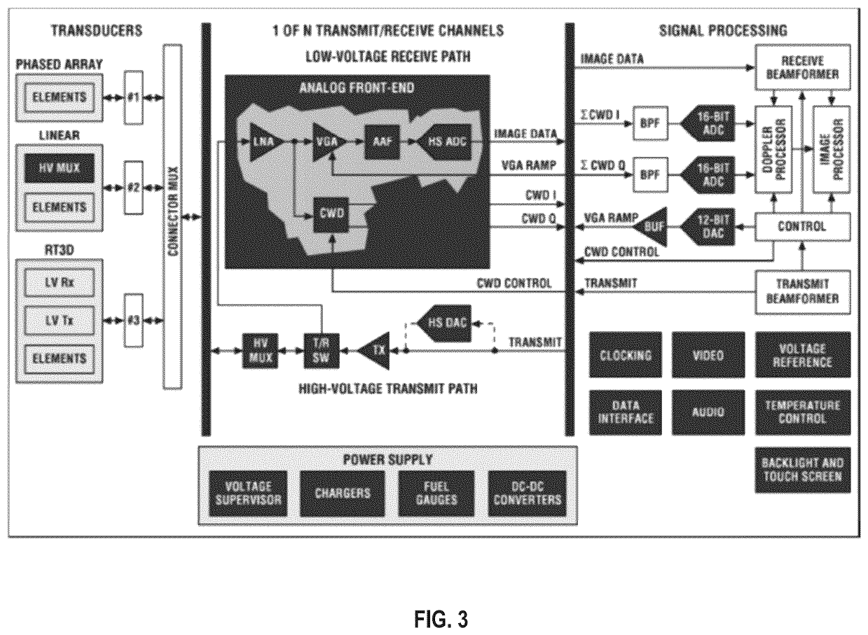

FIG. 3 illustrates an example computing environment of a measuring device comprising one or more components in accordance with some embodiments. In FIG. 3, the ultrasound transducers of the measuring device may communicate with the transmit/receive channels. The ultrasound transducers may be used to transmit and receive high frequency sound waves. The information detected from the ultrasound transducers may be processed through the transmit/receive channels to the signal processing system of the processing device of the measuring device. The signal processing system may be used to determine the high frequency sound waves to be transmitted by the ultrasound transducers, and also receive the high frequency sound waves that are received by the ultrasound transducers. The signal processing system may further process and analyze the received high frequency sound waves to extract data associated with the vital signs and internal parts of the physiological structure that the measuring device is placed against. The signal processing system may process and analyze the received high frequency sound waves by measuring the frequency of the received high frequency sound waves. The measuring device also includes a power supply that is used to power the measuring device.

In embodiments that use non-receiving transducers, the non-receiving transducers may be used to transmit high frequency sound waves. non-receiving transducers may be used interchangeably with ultrasound or acoustic transducers operated in a “transmit only” mode. The information detected from the impedance of the ultrasound transmitters may be processed through the transmit/receive channels to the signal processing system of the processing device of the measuring device. The receive channels may transmit data related to the impedance of the ultrasound transmitters. The signal processing system may be used to determine the high frequency sound waves to be transmitted by the ultrasound transmitters, and also receive the impedance data obtained from the ultrasound transmitters. The signal processing system may further process and analyze the received impedance data to extract data associated with the vital signs and internal parts of the physiological structure that the measuring device is placed against. The signal processing system may process and analyze the received impedance data by comparing data values, such that higher data values may indicate non-resonant internal structure and lower data values may indicate resonant internal structures. The measuring device may also include a power supply that is used to power the measuring device.

The measuring device may also include an accelerometer to detect when a physiological structure has fallen. For example, an elderly person may be susceptible to falling and severely injuring herself after a fall to where the elderly person is unable to help herself. The measuring device may also include additional components that may be used to detect and monitor particular attributes and conditions of a physiological structure. Additional components may be included in measuring devices according to the needs of the physiological structure using the respective measuring device. In this way, a measuring device may be customized to include any and all components that may be necessary to detect and monitor attributes relating to the vital signs, internal parts, and physical health of a physiological structure according to the needs of the physiological structure.

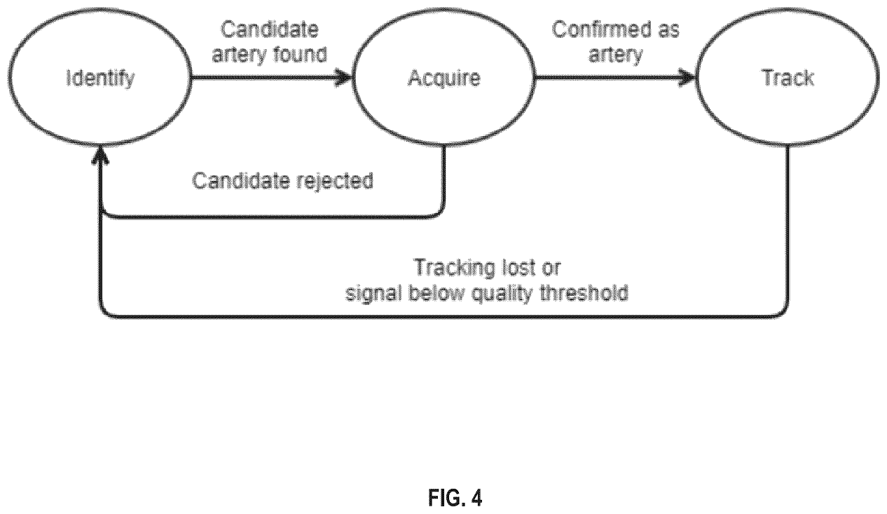

FIG. 4 illustrates an example process of a measuring device. The measuring device may identify various organs or anatomical structures identifiable by ultrasound. The measuring device may first identify vital signs of a physiological structure. The measuring device may determine if a blood vessel, such as an artery or vein, is found. The measuring device may analyze the vital signs to determine if the identified artery is actually an artery, or is the artery that is desired to be found and monitored. If the measuring device confirms that the correct artery is identified, the measuring device may determine the attributes of the artery (including but not limited to arterial wall thickness, vessel diameter, vessel radius, arterial resonance, arterial plaque thickness) and also continuously monitor the artery for a particular duration. In one example, the measuring device may apply LaPlace's Law or the LaPlace Theorem to measure resonance frequency in an artery, or blood vessel, to determine attributes of the artery such as its internal pressure or wall tension. Ultrasound arrays can be used to directly measure diameter. In anther example, impedance of the ultrasound transmitter may be used to find the resonance frequency of an artery, or blood vessel, which can then be used to determine attributes of the artery such as internal pressure or wall tension.

Vital signs associated with a blood vessel may include measurements of the blood vessel and attributes of the blood vessel. Measurements of the blood vessel may include the blood vessel's dimensions, i.e., blood vessel wall stiffness, blood vessel wall thickness, vessel radius, cross-sectional diameter, intima-media thickness, shape, circumference, etc. Attributes of the blood vessel may include resonance response of the blood vessel, the velocity of blood flow in the blood vessel, the detection of clots and other particles in the blood vessel, distance between the blood vessel and the monitoring device, clot burden, and plaque thickness of the blood vessel. The resonance response of the blood vessel may include resonant frequency of acoustic signals reflected by the blood vessel and/or the a determination of resonant frequency of the blood vessel based on the impedance of the ultrasound transmitter. The resonant frequency may correspond to a vibration of the blood vessel wall of the blood vessel cause by the reflected audio signals or may correspond to the resonant frequency that lowers the impedance of the ultrasound transmitter. Resonant frequency in a blood vessel may be used to determine attributes of the artery such as its internal pressure or wall tension. Audio arrays can be used to directly measure diameter of a blood vessel. Analyzing the vital signs of the identified blood vessel may determine the blood vessel type of the identified blood vessel. A blood vessel type may include, but may not be limited to, a vein, artery, carotid, subclavian, ascending aorta, descending aorta, axillary, brachial, radial, ulnar, palmar arch, renal, iliac, femoral, popliteal, tibial, anterior tibial, dorsalis pedis, posterior tibial, abdominal aorta, genicular, peroneal, plantar/dorsal arch, arcuate, or fibular. Other arteries not mentioned here may also be considered as types of a blood vessel.

Analyzing the vital signs of the identified blood vessel may also determine if the identified blood vessel is a blood vessel that is desired to be found and monitored, i.e., a target blood vessel. If the monitoring device confirms that the correct blood vessel is identified, the monitoring device may continuously monitor the blood vessel for a particular duration. The monitoring device may use vital signs associated with the identified blood vessel to determine other characteristics of the identified blood vessel.

In one example, the monitoring device may apply vital signs, such as a resonant frequency (f), a wall density (pS), a fluid density (pL), a radius-thickness product (γ), a wall thickness (h), a blood vessel/arterial radius (α), the arterial wall Young's modulus (E) and the Poisson's ratio of the wall (ν), of an identified blood vessel to a transformed Laplace's method that includes one or more transformed formulas, i.e., Equations (1), (2), (3), (4) and (5), to measure blood pressure (P) of the identified blood vessel. Some vital signs, such as resonant frequency (f), wall thickness (h) and blood vessel/arterial radius (α) may be measured by audio imaging from the monitoring device. Other vital signs, such as wall density (pS), fluid density (pL) and Poisson's ratio of the wall (ν) may be determined from a material database storing measurements of internal parts of physiological structures. Many variations are possible.

The blood vessel/arterial radius (α), resonant frequency (f), Poisson's ratio of the wall (ν), wall density (pS), fluid density (PL) and radius-thickness product (γ) of the identified blood vessel may first be applied to Equation (1) to determine the arterial wall Young's modulus (E).

$$E = ( \frac{8 {π}^{2} {a}^{7} f ( 1 - {v}^{2} )}{3 γ} ) ( \frac{4 {p}_{L} a ( f + a \frac{d f}{d a} ) + 5 {p}_{S} γ ( \frac{d f}{d a} )}{4 {a}^{7} - 3 {γ}^{2}} ) \qquad Equation ( 1 )$$

The wall thickness (h) and blood vessel/arterial radius (α) of the identified blood vessel may be applied to Equation (2) to determine the parameter α. The parameter α may be a dimensionless parameter that is used to represent the ratio between the wall thickness (h) and blood vessel/arterial radius (α) of the identified blood vessel.

$$α = \frac{h}{a} \qquad Equation ( 2 )$$

The parameter α determined from Equation (2), wall density (pS) and fluid density (pL) of the identified blood vessel may be applied to Equation (3) to determine the parameter p, which may be used to represent units of mass density per unit volume of the identified blood vessel.

$$ρ = α {p}_{S} + \frac{4}{5} {p}_{L} \qquad Equation ( 3 )$$

The arterial wall Young's modulus (E) determined from Equation (1), parameter p determined from Equation (3), resonant frequency (f), blood vessel/arterial radius (α) and Poisson's ratio of the wall (ν) may be applied to Equation (4) to determine the parameter D. The parameter D may be a dimensionless parameter that is used to simply the formula, i.e., Equation (5), to measure the blood pressure of the identified blood vessel.

$$D = 4 {π}^{2} ( 1 - {v}^{2} ) \frac{ρ {a}^{2} {f}^{2}}{E} \qquad Equation ( 4 )$$

The arterial wall Young's modulus (E) determined from Equation (1), parameter α determined from Equation (2) and parameter D determined from Equation (4) may be applied to Equation (5) to determine the blood pressure (P) of the identified blood vessel.

$$P = ( \frac{9 {α}^{4} - 5 ( 3 α + {α}^{3} ) D + 3 {D}^{2}}{- 4 ( 9 α - {α}^{3} ) + 12 D} ) E \qquad Equation ( 5 )$$

The monitoring device may also transmit, from the audio sensors, audio energy into an inanimate object. The monitoring device may receive, with the audio sensors, audio energy reflected back from internal parts of the inanimate object. The monitoring device may receive, with the audio sensors, audio energy reflected back from internal parts of the inanimate object. The audio energy received with the audio sensors may include information associated to internal parts of the inanimate object. The information associated to internal parts of the inanimate object may identify structures and characteristics of the inanimate object that are identifiable by audio.

The measuring device may monitor the vital signs and internal parts of a physiological structure until the measuring device is no longer placed against the physiological structure. The measuring device may monitor the vital signs and internal parts of a physiological structure until the measuring device loses power or loses signal in detecting the high frequency sound waves and/or impedance of the ultrasound transmitter. The measuring device may monitor the vital signs and internal parts of a physiological structure until the measuring device is moved to a location on the physiological structure that is unable to determine and monitor the particular vital signs and/or internal parts of the physiological structure.

In one example, the measuring device may detect the blood pressure of a blood vessel. To detect the blood pressure of a blood vessel, the measuring device may be placed against a physiological structure, such as a person, inanimate structure, or animal. The measuring device may be placed against any location of a physiological structure, such as an arm, leg, waist, abdomen, etc., that may contain a blood vessel. In one embodiment, the measuring device may be placed against the upper arm of a physiological structure to detect the blood pressure of the brachial artery. In one embodiment, the measuring device may be placed against the forearm of a physiological structure to detect the blood pressure of the radial artery. In another embodiment, the measuring device may be placed against the thigh/groin/pelvis of a physiological structure to detect the blood pressure of the femoral artery. In another embodiment, the measuring device may be placed against the wrist of a physiological structure to detect the blood pressure of the ulnar or radial artery. In another embodiment, the measuring device may be placed against the abdomen of a physiological structure to detect the blood pressure of the aorta. In another embodiment, the measuring device may be placed against the neck of a physiological structure to detect the blood pressure of a carotid artery. In another embodiment, the measuring device may be placed against the abdomen of a physiological structure, such as a human baby, to detect the blood pressure of the abdominal aorta. In another embodiment, the measuring device may be placed against the wall of a cylindrical thin shell, such as a rocket, to assess pressures of various internal fluids. In another embodiment, the measuring device may be placed against the wall of a cylindrical thin shell such as a pipe to assess internal water or oil pressures.

Clots and other particles in the bloodstream can be detected by the monitoring device because they behave differently from the regular cells making up the blood stream. Clots and other particles exhibit distinguishable fluid mechanics behavior. Namely, clots and other particles moving in the blood stream travel at a different speeds than the surrounding blood stream. Clots and other particles also have a tendency to occupy particular positions within a blood vessel as well as particular positions relative to each other. These three behavior patterns, (i) differential speed, (ii) relative position within the blood vessel, and (iii) relative position to other particles in the blood flow, provide for the detection of clots and their properties.

Individual clots or other particles moving in a bloodstream may appear as “events” in a Doppler spectrum as they pass through the range of an audio probe. Measuring the Doppler shift relative to the frequency of the blood flow in the portion of the imaged blood vessel provides a strong reference point. In other words, the Doppler shift is measured relative to a central frequency. Therefore, the presence of a clot or other particle moving in the blood flow can still be detected with a high level of confidence even if there are variations in the blood flow. For instance, changes in the blood flow may be present depending on which portion of a blood vessel is measured within a physiological structure, whether the physiological structure has eaten recently, and other factors. The clot detection is also reliable in different physiological structures who may have differing blood flow baselines.

Additionally, the magnitude and frequency of an observable Doppler shift may also depend on the size of the particle or blood clot. Therefore, a Doppler signal may reveal multiple pieces of information about particles in the blood. For instance, it may reveal not only their existence but also properties such as frequency, size, position, and other factors. These additional factors may assist in distinguishing clots and other factors from noise.

The detection of clots and other particles in the bloodstream of a blood vessel is desirable because it would enable doctors to identify and treat conditions caused by the clots and other particles early, before a patient begins to experience adverse health conditions, including a stroke, pulmonary embolism, deep vein thrombosis, and other conditions. The monitoring device may detect clots and other particles in the bloodstream of a blood vessel when the monitoring device is placed against a surface of a physiological structure containing the blood vessel. The monitoring device may obtain cross-sectional and longitudinal B-mode imaging of the blood vessel. The monitoring device may use the cross-sectional and longitudinal B-mode imaging of the blood vessel to visually or algorithmically detect clots and other particles in the blood vessel. The monitoring device may use an audio sensor with a color Doppler technique to detect clots and other particles in the bloodstream of a blood vessel. The color Doppler technique may use pulse-wave Doppler with short pulses to create an image sequence of blood flow in a target region of a blood vessel. The image may contain information about the presence and properties of particles, including blood clots, suspended in the bloodstream. The monitoring device may identify when the size of clots and other particles are large. The monitoring device may send a notification when large clots and other particles have been identified. The notification may be sent automatically to authorized individuals, such as doctors, nurses, medical practitioners, etc. The notification may be sent automatically to a system that is used to monitor the health of the physiological structure and process medications for the physiological structure.

Machine learning may be used to extract the relevant information from an image sequence of a blood flow. Machine learning techniques may also be used to identify and interpret patterns consistent with events from the Doppler spectrum, thereby confirming the presence of a clot or other particle in the bloodstream of a blood vessel. Machine learning techniques may further be used to interpret event patterns to characterize the size and frequency of different particles or clots in the bloodstream. A machine learning model may be trained using collected data comprising sample image sequences of blood flows and known targets. Known targets may be the confirmed size or frequency of a particle or blood clot in a particular sample. Known targets may also be other parameters. In an embodiment, a known target may be the risk that a clot will cause a particular health problem within a particular time frame. In an embodiment, the machine learning model may be a neural network.

In one embodiments, when clots and other particles are detected in a blood vessel and the clots and other particles have been identified to not be large in size, machine learning algorithms may be applied for greyscale identification of microclots and other small particles in the blood vessel. The monitoring device may send a notification when microclots and other small particles are not identified. The notification may be sent automatically to authorized individuals, such as doctors, nurses, medical practitioners, etc. The notification may be sent automatically to a system that is used to monitor the health of the physiological structure and process medications for the physiological structure. When microclots and other small particles have been identified to be in the blood vessel, the machine learning algorithms may be used to determine the size, frequency and other characterized attributes of the microclots and other small particles in the bloodstream of the blood vessel. The monitoring device may send a notification of the characterized attributes to a system and/or to authorized individuals.

In one example, the monitoring device may detect clots and other particles in the fluid of a blood vessel. The monitoring device may first collect data from the audio energy received by the audio sensors. The data collected from the audio energy may include information associated to a blood vessel. The monitoring device may use the information to determine the relative speed of blood flow in the blood vessel at various positions of the blood vessel. Variations in the speed of blood flow in a blood vessel may indicate a detection of clots and other particles in the blood vessel. The monitoring device may also use the information to determine changes in the frequency wave relative to the speed of blood fluid in the blood vessel. Changes in the frequency wave relative to the speed of the blood fluid in the blood vessel may also indicate a detection of clots and other particles in the blood vessel. If the monitoring device determines there are variations in the speed of blood flow in a blood vessel, the monitoring device may use the various speeds of blood flow in the blood vessel to determine the relative position of the clots and other particles in the blood vessel. Clots and particles may affect the speed of the blood fluid in the blood vessel under certain conditions, including at the positions where the clots and other particles are located in the blood vessel and causing blockage to the blood flow. The speed of blood fluid and the position of clots and other particles in a blood vessel may be affected by the size, shape, and other properties of the clots, particles and blood vessel. The changes in the speed of the blood fluid and the frequency wave relative to the speed of the blood fluid may be used to determine the existence, size and frequency of clots and other particles in the blood vessel.

The monitoring device may use the information associated to the vital signs, blood pressure and other characteristics of an internal part, such as a blood vessel, to determine a diagnosis of a medical condition with the physiological structure. Based on the determination of a diagnosis of a medical condition and information associated to the vital signs, blood pressure and other characteristics of an internal part of a physiological structure, the monitoring device may determine if a medication should be made for the physiological structure. The monitoring device may determine a diagnosis of a medical condition and that a medication needs to be made for the physiological structure by comparing information currently obtained from the audio sensors to information previously obtained and/or stored associated to the same internal part.

The monitoring device may obtain pre-determined information associated to the vital signs, blood pressure and other characteristics of an internal part, such as a blood vessel. The monitoring device may compare the pre-determined information associated to the internal part with presently determined information of the internal part. By comparing the pre-determined information with the presently determined information of the internal part, the monitoring device may determine any variations in the vital signs, blood pressure and other characteristics of the internal part.

The monitoring device may display information associated to an internal part, such as a blood vessel, on a graphical user interface (GUI). A GUI may include a monitor, screen, projection, television, etc. In one example, the monitoring device may display presently determined information of vital signs, blood pressure and other characteristics associated to a blood vessel on a GUI for an individual to view. In another example, the monitoring device may display both presently determined information and pre-determined information associated to a blood vessel on a GUI to display any differences between the information.

In one example, the monitoring device may determine the current blood pressure of a blood vessel using information associated to the vital signs and other characteristics of the blood vessel that are obtained from audio energy received by the audio sensors. The monitoring device may compare the current data of blood pressure, vital signs and other characteristics of the blood vessel to pre-determined data of blood pressure, vital signs and other characteristics of the blood vessel. When the monitoring device determines there is a variation between the current data and the pre-determined data of the blood vessel, such as a variation in the blood pressure, the monitoring device may determine a medication needs to be made for the physiological structure, such as a person. When the monitoring device determines a medication needs to be made for the physiological structure, the monitoring device may generate a report. The report may include information associated to the blood vessel, including the current vital signs, blood pressure and other characteristics of the blood vessel. The report may include a medication prescription for the determined medication. The report may be sent automatically to authorized individuals, such as doctors, nurses, medical practitioners, etc. The report may be sent automatically to a system that is used to monitor the health of the physiological structure and process medications for the physiological structure.

In one example, the monitoring device may determine that the current value of blood pressure of a blood vessel of a person is above the pre-determined value of blood pressure of the blood vessel of the person. The monitoring device may determine a medication needs to be made for the person based on the determination of the difference between the current value and pre-determined value of blood pressure of the blood vessel. The monitoring device may then generate a medication prescription for the person. The medication prescription may include information of the variation in the blood pressure of the blood vessel of the person and the type of medication that needs to be made. The monitoring device may send the medication prescription to a system associated with the monitoring device for a medication to be made according to the medication prescription.

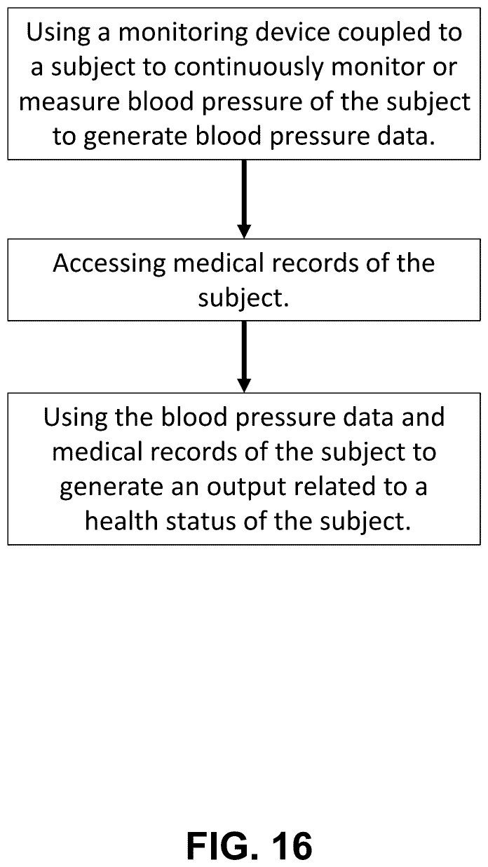

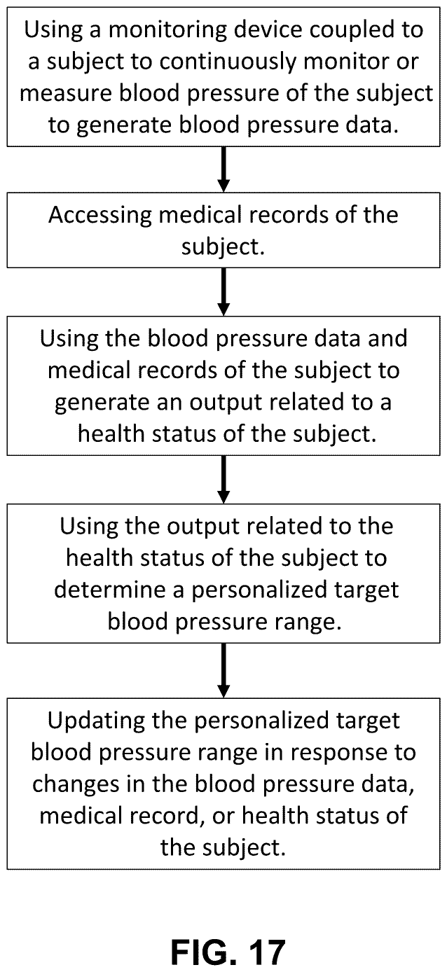

In an example, the present disclosure may provide methods including using a monitoring device coupled to a subject to continuously monitor a blood pressure of the subject to generate blood pressure data; and using at least the blood pressure data and medical record data of the subject to generate an output related to a health status of the subject. In an example, the present disclosure may provide methods including using a monitoring device comprising a sensor coupled to a subject to monitor a blood pressure of the subject to generate blood pressure data; and using the monitoring device to analyze electronic medical records of the subject and generate an output related to a health status of the subject from at least the electronic medical records and the blood pressure data. As shown in FIG. 16, the monitoring device may be coupled to a subject to continuously monitor or measure a blood pressure of the subject to generate blood pressure data. The subject's medical records may be accessed either by the subject, a medical practitioner, or by the monitoring device. The blood pressure data may be used in combination with the medical records of the subject to generate an output related to a health status of the subject. The output may be generated by the monitoring device upon input by the subject or a medical practitioner. Alternatively, the monitoring device may access the medical records and automatically generate an output related to the health status of the subject based at least in part on the blood pressure data and medical record. In an example, the present disclosure may provide methods including automatically generating a personalized target blood pressure range for a subject from continuous blood pressure measurement data obtained from a monitoring device coupled to the subject and medical record data of the subject. As shown in FIG. 17, the output related to health status may be used to determine a personalize target blood pressure range of the subject. The personalized target blood pressure range may be more accurate than personalized target blood pressure ranges determined using non-continuous or cuff-based blood pressure devices. The monitoring device may update the personalized target blood pressure range, either automatically or after a prompt or input from the subject or medical practitioner, in response to a change in blood pressure data, medical record, or health status of the subject. In some cases, the monitoring device may determine the current blood pressure of a blood vessel of a person as described elsewhere herein. The monitoring device may continuously monitor the blood pressure with the blood vessel of the person. The blood pressure measurements determined by the monitoring device may be combined with data from a patient's medical record, which may be a paper or electronic medical record. The monitoring device may access and analyze patient health record data (e.g., electronic medical records) from one or more sources. The monitoring device may be configured to automatically generate personalized target blood pressure ranges from continuous blood pressure measurement data and medical record data of the subject. The monitoring device may be configured to automatically or may automatically update the personalized target blood pressure range in response to changes in the blood pressure measurement data or the medical record data. The patient health record data may comprise but is not limited to patient demographic information (e.g., age, gender, etc.), longitudinal blood pressure measurements, a diabetes status of the patient, prior heart disease diagnoses, historical blood pressure readings, medication records, lab results, or the like. The monitoring device may analyze the current blood pressure of a blood vessel and patient medical record data to determine a status of the patient's health. The monitoring device may analyze the current blood pressure of a blood vessel and patient medical record data to determine whether the patient's current blood pressure is too high, too low, or within a normal range. The monitoring device may analyze the patient's health record data to determine a range for high blood pressure, low blood pressure and normal blood pressure based in part on a health status or disease status of the patient. Health status may include stress, fitness, nutrition, sleep, or any combination thereof. In an example, a measuring device may monitor an individual's stress levels. In another example, a measuring device may monitor an individual's fitness. In another example, a measuring device may monitor an individual's nutrition. In another example, a measuring device may monitor an individual's sleep (e.g., duration, quality, etc.). Measured indicators of health status may be monitored over time. Measured indicators of health status may be integrated into one or more health models to obtain data related to the overall health of an individual. In an example, the monitoring device may use the blood pressure of the subject and medical record data to determine a health status of the subject. For example, the monitoring device may provide a blood pressure alert to the subject or an external party upon the monitoring device determining that the blood pressure of the subject is below a first blood pressure alert threshold of the blood pressure alert thresholds or above a second blood pressure alert threshold of the blood pressure alert thresholds. In another example, the monitoring device may lower the threshold for high blood pressure for elderly patients. In another example, the monitoring device may lower the threshold for high blood pressure during the first or second trimester of pregnancy. In another example, the monitoring device may monitor changes in blood pressure during pregnancy and alert if the patient's blood pressure does not decrease in the second trimester or if the rate of blood pressure increases too rapidly after 20 weeks of gestation. The monitoring device may compare the current data of blood pressure, vital signs and other characteristics of the blood vessel to previous blood pressure readings for the patient or patients of similar age, gender, and medical background.

In some cases, when the monitoring device determines the current blood pressure of a blood vessel is not within a normal or healthy range, the monitoring device may perform one or more actions. The one or more action may include but are not limited to: delivering a notification or alert to the patient or a third party (e.g., monitoring service, healthcare provider, etc.), updating the patient's medical record, ordering a medication, directing that a treatment be delivered, delivering a treatment, or perform an emergency protocol. In some cases, the monitoring device may be configured to alert one or more of the patient, a medical professional, emergency service, or emergency contact. In some cases, the monitoring device may be configured to provide the patient with instructions or recommendations for improving their blood pressure. In some cases, the monitoring device may determine a medication needed to treat the patient. When the monitoring device determines a patient needs to be treated with a medication, the monitoring device may generate a report. The report may include information associated to the patient, including the current vital signs, blood pressure and other characteristics determined from the blood vessel.

In some cases, the monitoring device may be configured to dynamically adjust threshold values and range definitions for high, low, and normal blood pressure. In some cases, the thresholds and ranges are dynamically adjusted in real time based at least in part on the patient's medical records. The monitoring device may be configured to receive and weigh relevant data such as recent longitudinal blood pressure measurements, diagnosed conditions (e.g., diabetes, heart failure, chronic kidney disease (CKD), etc.), current medications and recent medication changes, clinician-specified targets, demographic factors (e.g., age, pregnancy status), and pertinent laboratory values. In some cases, the monitoring device may compute personalized thresholds as new record data are received or updated. These adjustments may be made using one or more of rule-based logic, model-based risk scoring, or a combination thereof. In some cases, each computed threshold is linked with provenance and rationale metadata so clinicians and patients can review reasoning behind threshold value setting and changes.

The monitoring device may be configured to compute and store a patient-specific baseline blood pressure profile. In some cases, the patient-specific baseline blood pressure profile is derived from a time-windowed aggregation of prior validated measurements. In some cases, the patient-specific baseline blood pressure profile is based at least in part on a median, mean, interquartile range, or standard deviation of the last N valid home or clinic readings. In some cases, the baseline profile may be annotated with location data indicating the environment where the reading was collected. In some cases, the location data determines whether the reading was collected while the patient was at home, a doctor's office, or other location. In some cases, the baseline profile may be annotated with measurement context to determine an activity status of the patient during the reading, (e.g., resting, running, working out, post-exertion, first reading of day, etc.). In some cases, the baseline profile may be annotated with measurement quality metadata extracted from the audio signal (e.g., signal-to-noise ratio, presence of motion artifact, heart rate concordance, etc.). In some cases, the monitoring device may weight historical readings according to recency, measurement quality, and provenance when computing the baseline. In some cases, the monitoring device may apply a higher weight to clinic-validated measurements and recent stable home readings.

In some cases, the monitoring device may implement a tiered alerting framework that maps measured values and contextual risk to discrete alert levels. In some case the alert level may include an informational level, advisory level, urgent level, or emergency level. Each alert level may be associated with pre-configured actions or workflows and notification channels. For informational alerts the system may log the event. The monitoring device may send a low-priority message to the patient's application with self-care guidance. For advisory alerts the system may automatically schedule or suggest a clinician visit. The monitoring device may include a summary of recent trend data. For urgent alerts the system may send a prioritized message to a treating clinician's inbox or to on-call personnel. The urgent alert may include an attached trend graph, provenance metadata, and recommended next steps. For emergency alerts the system may attempt to contact emergency services, simultaneously notify emergency contacts, and provide location and critical readings to responders.

In some cases, the decision to perform each action may be governed by explicit business rules, clinician-configured thresholds, or a predictive risk model. In some cases, the monitoring device may comprise one or more predictive models may compute a probability that the current measurement pattern will lead to an adverse event within a defined time period (e.g., 1 hour, 12 hours, 24 hours, or 72 hours). In some cases, the monitoring device may translate that probability into an alert level using calibrated thresholds. The model outputs may be accompanied by decision metadata so that clinicians can understand why the system recommended escalation.

In some cases, the system may require confirmation of abnormal readings via one or more validation to prevent spurious alerts. The one or more validation steps may include: prompting the patient to remeasure after standardized rest, automatically re-running an artifact detection algorithm on the audio signal, or cross-validating with contemporaneous vitals from other sensors. The system may suppress escalation if validation fails or may tag such events as low-confidence alerts for clinician review.

In another example, the measuring device may be placed against a physiological structure, such as a person, inanimate structure, or animal, to monitor and assess an organ, such as the heart, lungs, kidneys and liver, of the physiological structure. The measuring device may be placed against a particular location of the physiological structure, such as the chest, abdomen, back, etc., depending on the organ to be monitored. In one embodiment, the measuring device may be placed against the chest of the physiological structure to monitor the heart and assess for any pathology with the heart. In another embodiment, the measuring device may be placed against the back of the physiological structure to monitor the lungs and assess for any pathology with the lungs. In another embodiment, the measuring device may be placed against the abdomen of the physiological structure to monitor the liver and assess for any pathology with the liver.

The measuring device may be attached to an accessory that can allow the measuring device to be placed against a physiological structure and remain in a particular location. The accessory to attach to the measuring device may be an elastic material, such as a strap or band, that can be used to wrap the measuring device against a location of the physiological structure and hold the measuring device in place. The accessory may also be an adhesive material, such as a sticker or adhesive patch, to stick the measuring device against a particular location of the physiological structure and hold the measuring device in place. The measuring device may be integrated into another wearable device, for example, the measuring device may be integrated into a watch or sweatband. Keeping the measuring device in the same position against the physiological structure may allow the measuring device to more accurately detect the blood pressure of a blood vessel.

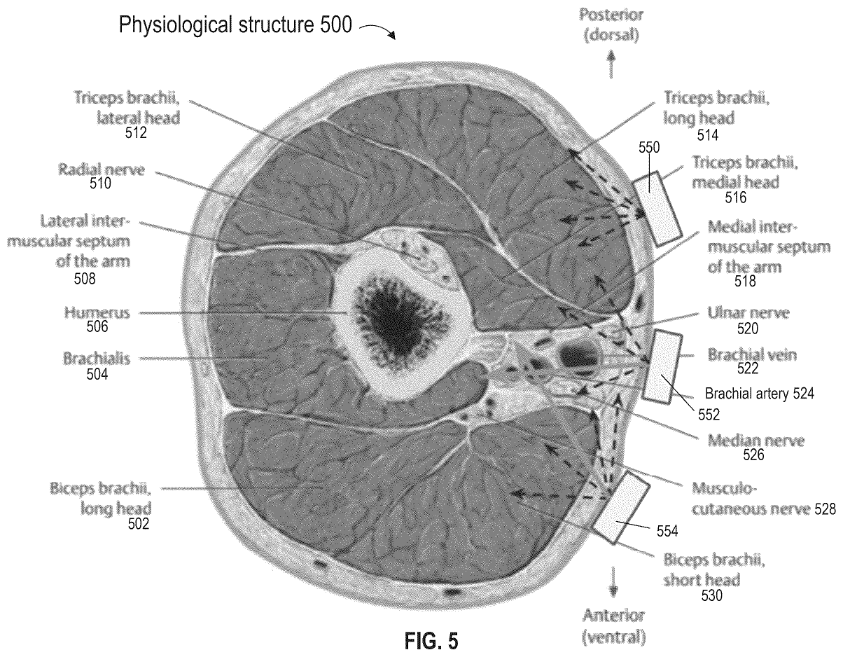

FIG. 5 illustrates an example image of a physiological structure 500 that is being monitored by the acoustic transducers 550, 552, and 554. Acoustic transducers 550-554 may be part of a measuring device of the presently disclosed technology. For example, acoustic transducers 550-554 may be implemented on a wearable arm band or cuff of the measuring device.

As depicted, physiological structure 500 comprises a cross-sectional view of a person's arm. Physiological structure 500 comprises various internal physiological sub-structures which can be monitored/detected by the measuring device. Such internal physiological sub-structures include a biceps brachii (long head) 502, a brachialis 504, a humerus 506, a lateral inter-muscular septum of the arm 508, a radial nerve 510, a triceps brachii (lateral head 512), a triceps brachii (long head) 514, a triceps brachii (medial head) 516, a medial inter-muscular septum of the arm 518, an ulnar 520, a brachial vein 522, a brachial artery 524, a median nerve 526, a musculocutaneous nerve 528, a biceps brachii (short head) 530, etc.

In FIG. 5, the measuring device may be placed at a location of physiological structure 500 where the acoustic transducers of the measuring device (e.g., acoustic transducers 550, 552, and 554) are pressed flat against the surface of the physiological structure. The acoustic transducers may transmit low or high frequency sound or audio waves into physiological structure 500 in multiple directions. The measuring device may use the low or high frequency sound or audio waves to detect and identify vital signs and internal physiological sub-structures of physiological structure 500. As alluded to above, internal physiological sub-structures may include blood vessels (i.e., veins and arteries), organs (i.e., heart, lungs, liver, kidneys, small intestine, large intestine, stomach, brain, etc.), bones, tissues, muscles, tendons, etc. Internal parts may also include various pathologies including, but not limited to, a fracture, an abscess, a tumor, cellulitis, stones, etc. The measuring device may also use the low or high frequency sound waves to measure, induce, or detect frequency response in a blood vessel. The measuring device may further use the low or high frequency sound waves to determine properties of the blood vessel and other internal parts of the physiological structure. The measuring device may apply a transformed LaPlace's method, directly image through audio, or calculate from obtained transducer impedance to measure the frequency in a blood vessel to determine attributes of the blood vessel, such as its diameter.

To monitor vital signs and internal parts of a physiological structure, the measuring device may first generate a machine learning (ML) model. The ML model may be generated using initially determined vital signs and other characteristics of internal parts of a physiological structure. The initial vital signs of internal parts may be determined from information associated with the audio energy received by the acoustic transducers. The other characteristics of internal parts may be determined from the initial vital signs. Once the ML model is generated, newly gathered information associated to vital signs and internal parts may be applied to the ML model to determine if any changes have occurred with the vital signs and internal parts of the physiological structure.

In one example, the measuring device may monitor the blood pressure of a blood vessel in a physiological structure, a ML model may be generated using initially determined or pre-determined vital signs and other characteristics of the blood vessel. The initially determined vital signs and other characteristics of the blood vessel may be determined by the measuring device from the audio energy and audio signals received by the acoustic transducers that initially identified the blood vessel. The pre-determined vital signs and other characteristics of the blood vessel may be stored in a database of a system for the measuring device to extract. When the measuring device obtains new vital signs and other characteristics of the blood vessel, the new vital signs and characteristics may be applied to the ML model to determine if any changes have occurred with the vital signs and other characteristics of the blood vessel, including the blood pressure of the blood vessel. Determining changes in the vital signs and other characteristics of internal parts, such as a blood vessel, of a physiological structure may detect and diagnose a medical condition present in the physiological structure.

Medical conditions that may be detected in a physiological structure by the measuring device includes congenital cardiac disorder, limb ischemia, cardiovascular anomalies, preeclampsia, sepsis, infection of continuous temp, hypoxia, pneumonia, intubation, complications with pulse oximetry, tachycardia, hypotension, hypertension, internal bleeding, hemorrhage, chronic lung disease, risk of seizures, sleep apnea, postural orthostatic tachycardia syndrome, low blood pressure, low glucose level, deep vein thrombosis (DVT), stroke, pulmonary embolism (PE), superficial thrombophlebitis, blood clotting, tension pneumothorax, supraventricular tachycardia (SVT), idiopathic intermittent atrial fibrillation, angina, myocardial infarction (MI), hyperglycemia, and diabetic ketoacidosis (DKT).

In some cases, a monitoring device can accurately and continuously track blood pressure (BP) variability and can detect characteristic BP patterns associated with disease, disease progression, or response to treatment. In some cases, the BP variability or patterns associated with changes in BP may be usable to aid in the diagnosis of a disease, monitor progression of a disease, monitor treatment of a disease, or any combination thereof. In some cases, the device is configured to analyze time-series BP data and to detect disease-specific patterns. In some cases, the device can assist with diagnosis and monitoring of many conditions by precisely characterizing BP variability, isolated BP values, or combinations of these signals. In some cases, the ability to capture, quantify, and longitudinally track BP variability over time can provide clinicians and researchers with relevant data for diagnosing a condition or evaluating a treatment.

In some cases, the device can detect BP patterns related to Parkinson's disease, including large intraday swings, frequent orthostatic drops, postprandial hypotension, and episodes of nocturnal hypertension. The ability to track and analyze BP variability continuously over extended time windows can capture events and patterns that single-point measurements may miss. In some cases, the device can reliably timestamp systolic and diastolic values, record measurement-quality metadata (for example motion index, posture, sensor signal-to-noise ratio (SNR)), and preserve contextual markers (for example time of day, meal or medication times, activity). In some cases, the device can measure how much a blood pressure reading deviates from a previous reading or baseline reading. In some cases, the device measures how often a patient's BP deviates from a previous reading or baseline reading, and identifies real physiological changes from measurement errors. For example, the device can report the peak-to-trough change in systolic blood pressure, count episodes above 200 mmHg, and track how often blood pressure drops when a person stands.

In some cases, the device can sample at high temporal resolution, and in some cases, sampling is beat-to-beat. In some cases, sampling is minute-level, hourly, daily, or weekly depending on the clinical use case. In some cases, the device can capture auxiliary signals (for example audio-derived pulse features, photoplethysmography (PPG), accelerometry, and posture) that enable artifact rejection, motion compensation, and physiologic cross-validation. In some cases, each measurement is tagged with provenance and quality metadata. In some cases, the device can perform calibration routines, adaptive filtering, and multi-sensor fusion to reduce bias and variance in the recorded BP values.

In some cases, once accurate time-series datasets are available, a processing module can compute a set of blood pressure variability (BPV) metrics over configurable time windows (for example hourly, daily, weekly). In some cases, the processing module can compute metrics including standard deviation, coefficient of variation (CV), average real variability (ARV), range and interquartile range, time-of-day stratified statistics (for example day/night and orthostatic epochs), slope and rate-of-change, and frequency of excursions outside personalized thresholds. In some cases, the device can aggregate validated time-series into windowed metrics (for example daily/weekly ARV, SD, CV, night/day differences, orthostatic response distributions) and can compare them to patient-specific baseline data.

In some cases, the processing module can use measurement-quality metadata and auxiliary signals to distinguish true physiological variability from artifact. In some cases, the device can apply patient-specific contextual information (for example medication timing, recent dose changes, comorbidities, and symptom logs such as dizziness or syncope) or electronic health record (EHR) data to determine clinically meaningful variability versus expected pharmacologic or situational effects. In some cases, the device can apply risk models or anomaly-detection algorithms to flag atypical variability patterns. In some cases, annotated BPV metrics and time-series are retained with provenance and algorithm versioning for auditability.

In some cases, the device can generate detailed reports including labeled blood pressure time-series, counts and sizes of extreme events, and records of symptoms and medications. For example, a report can show when big BP spikes or drops occurred alongside notes about dizziness or recent doses of medicine. In some cases, because the device can find abnormal patterns even in early-stage or symptom-free patients, it can support earlier diagnostic follow-up and more targeted evaluation.

In some cases, identification of one or more BP variability patterns can trigger a diagnosis or next steps in a diagnosis workflow. For example, in response to repeated large swings in systolic BP (about 50-100 mmHg), high nighttime BP with low daytime BP, frequent drops when standing, or other unusual patterns, the device may generate advisory reports recommending tests or a referral. In some cases, the device may provide a preliminary diagnosis based on the one or more BP variability patterns. For example, the device may recommend autonomic function tests or a neurology referral because autonomic cardiovascular dysregulation can be an early sign of Parkinson's-related autonomic failure.

In some cases, the device can measure and record objective, repeatable endpoints to track disease progression and treatment effects. In some cases, the device can perform longitudinal trend analysis to quantify whether autonomic instability is worsening or improving with treatment. For example, the device can report change-from-baseline in weekly ARV, reductions in the frequency of hypertensive or hypotensive excursions, or stabilization of nocturnal dipping. In some cases, the device can enforce standardized sampling protocols and use versioned algorithms to derive variability endpoints that support centralized, auditable comparisons across subjects and timepoints in clinical trials. In some cases, because the device can detect treatment-emergent hypertensive peaks or worsening variability early, it can enable timely medication adjustments and can enhance patient safety by surfacing risks such as falls or stroke that intermittent clinic measurements might miss.

In some cases, a clinician can use BPV measured by the device as a diagnostic and monitoring biomarker. In some cases, the device can detect Parkinsonian autonomic impairment patterns such as excessive short-term variability, large orthostatic changes, impaired nocturnal dipping, increased frequency of transient hypotensive or hypertensive excursions, or progressive increases in variability over weeks to months. In some cases, the device can link detected patterns to concurrent symptoms (for example dizziness, syncope, orthostatic intolerance) and medication histories (for example dopaminergic therapy, antihypertensives). In some cases, the platform can generate advisory reports that summarize variability metrics, illustrate annotated time-series, and recommend autonomic function testing or specialist referral. In some cases, the platform can quantify pre-treatment baselines, detect treatment-emergent changes, and provide validated variability-derived endpoints (for example weekly ARV or change-from-baseline CV) with documented algorithm versions and confidence intervals to support safety monitoring, efficacy analyses, and regulatory submissions.

In some cases, the device can tag each measurement with provenance and quality metadata and in some cases can use that metadata to allow clinicians to audit, filter, or reprocess data. In some cases, the device can export annotated BPV metrics, time-series, and associated metadata for integration with clinician workflows and EHRs; in some cases, exports can include algorithm version identifiers and confidence intervals to support regulatory submissions or centralized analyses.

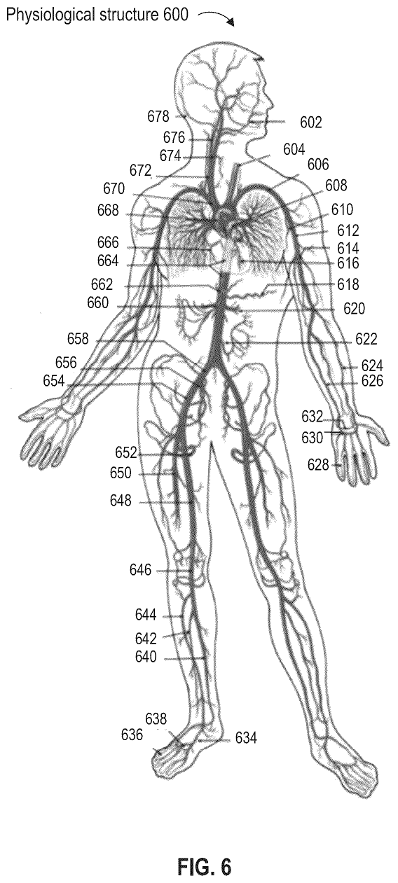

FIG. 6 illustrates an example image of a physiological structure of a human body with various internal parts of the human body that may be identified and monitored using the measuring device. In one example, a blood vessel may include an artery or vein in the physiological structure. A blood vessel whose blood pressure is detected by the measuring device may include a vein, artery, carotid, subclavian, ascending aorta, descending aorta, axillary, brachial, radial, ulnar, palmar arch, renal, iliac, femoral, popliteal, tibial, anterior tibial, dorsalis pedis, posterior tibial, abdominal aorta, genicular, peroneal, plantar/dorsal arch, arcuate, fibular, or others. The measuring device may be placed against a particular location of a physiological structure to detect the blood pressure of a particular blood vessel, based on any number of factors, including the type of physiological structure (i.e., adult person, infant person, adult animal, infant animal, etc.) and the blood vessel of interest. Keeping the measuring device in the same position against the physiological structure may allow the measuring device to more accurately detect the blood pressure of the blood vessel of interest.

While perfect imaging of an internal part, such as a blood vessel, is not necessary for the purpose of detecting and monitoring vital signs of a person, having imagery of a better quality may allow for improved and faster identification and measurements of the internal part.

An ultrasound coupling medium may be placed on each ultrasound sensor. The ultrasound coupling medium may include a pad of a lubricant substance, such as silicone, gel, or hydrogel, which functions as an acoustic-impedance matching layer. The ultrasound coupling medium may allow clearer imaging of an internal part, such as a blood vessel, in comparison to imaging of the blood vessel obtained from using ultrasound sensors without an ultrasound coupling medium.

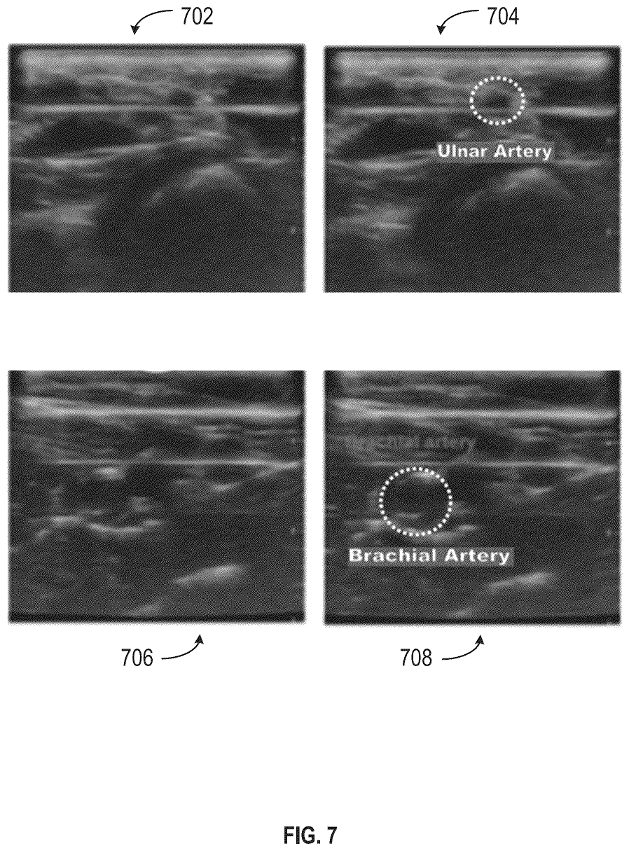

FIG. 7 illustrates example images of an internal part of a physiological structure that are generated by the measuring device. The example images show two sets of images taken at different locations of a physiological structure to detect and monitor different internal parts of the physiological structure. The example images show examples of different internal parts, such as an ulnar artery and brachial artery, that may be detected and monitored by the measuring device.

Further, the measuring device may use the collected data of a person's vital signs and internal parts, in combination or separately, with one or more measurements and produced images of the internal parts to generate an vessel location model. In one example, the vessel location model may be used to determine an accurate location of a blood vessel in the physiological structure. For example, the vessel location model may use conventional image recognition routines to locate, based on contrast changes, a blood vessel within the field-of-view of the ultrasound image from any given transducer. The ultrasound transducers may be located in proximity to each other such that neighboring transducers have overlapping fields-of-view. In such cases where a blood vessel falls within neighboring transducer fields-of-view, the vessel location model may evaluate which transducer's field-of-view has an image of the blood vessel with the highest signal-to-noise and/or image contrast. The transducer with the highest signal-to-noise and/or image contrast will then be activated and the neighboring transducers may be temporarily deactivated to save power. In some embodiments, neighboring transducers may remain powered on, or may be powered on at a regular interval to confirm that the primary active transducer still contains the best image of the blood vessel.

In some examples, the vessel location model may use ML (including but not limited to implementations of a “You Only Look Once” or “YOLO” convolutional neural net models), template tracking, and traditional computer vision techniques to ascertain a vessel's location. The vessel location model may reference a database of prior measurements of anatomy to bound its predictions.

The collected data, measurements, images, and location of the blood vessel may be displayed and viewed on a screen. The screen may be on the measuring device and/or on another device that is associated with the measuring device.

After the measuring device has generated an vessel location model for a particular internal part, such as a blood vessel, the measuring device may monitor the particular internal part. In one example, a particular blood vessel may be monitored by continuously having the measuring device placed against the physiological structure at a location where the blood vessel is located. In another example, a particular blood vessel may be monitored by periodically placing the measuring device against the physiological structure at a location where the blood vessel is located. By having the measuring device placed against the physiological structure at a location where the particular blood vessel is located, the measuring device may obtain new data on the blood vessel. The measuring device may use the new data to determine any changes to or associated with the particular blood vessel, including any changes to other organs, bones, tissues, etc. of the surrounding physiological structure. In one embodiment, the measuring device may compare the new data against the generated vessel location model for the particular blood vessel to determine any changes to the particular blood vessel.

Using the measuring device to monitor vital signs and internal parts, such as blood vessels, for any changes may allow the determination of any issues occurring in the physiological structure. In one embodiment, the measuring device may be used on a person to detect and monitor arterial lines for beat-to-beat monitoring to determine if any changes are occurring with the person's blood pressure, and make adjustments to vasoactive medications, such as a pressor, based on data on the blood pressure. The measuring device may be used in substitution of an arterial catheter in monitoring blood pressure, or be used in combination with an arterial catheter for multiple arterial blood sampling and allow for earlier removal of the arterial catheter when arterial blood samples are no longer needed.

In an embodiment, the measuring device may obtain various vital sign measurements of a person. The measuring device may also be used to continuously obtain vital sign measurements of a person and automatically send the data to associated devices. With the measuring device being able to obtain various vital sign measurements, it may replace the use of multiple devices, such as a blood pressure cuff and pulse oximeter, where each are needed to measure a single type of vital sign. Being able to use the measuring device in replacement of multiple devices and tools may allow for more ease in obtaining and monitoring measurements of a person and more quickly determine a diagnosis and treatment for the person. Using the measuring device may also allow for accurate, noninvasive readings and monitoring of a person's vital signs, compared to other devices. Using a single measuring device to obtain and monitor various vital signs may be less problematic and allow for obtaining faster results than using multiple different devices and tools, especially in emergency situations, where time is critical to saving a person who is in a life-threatening condition.

The measuring device may be portable and may run on battery, which may be chargeable and/or replaceable. The measuring device may be used in conjunction with other devices, such as computers, monitors, phones, tablets, etc. The measuring device may connect to other devices via a wired or wireless connection, such as Bluetooth. With the measuring device being portable, it may be used to obtain measurements of vital signs and internal parts of a physiological structure in any situation and location.

The monitoring device may send an alert when the monitoring device is low on battery. The monitoring device may send an alert when there is an issue with the communication connection it has with another device. The monitoring device may send an alert when there is an issue with a function of the monitoring device, such as an issue with the audio sensors. The monitoring device may send an alert to another device that it is connect to. The monitoring device may send an alert to a system, application, or platform that it is connect to. The monitoring device may display an alert on an GUI with a message or symbol indicating any issue that is occurring with the monitoring device. Many variations are possible.

In another example, emergency responders, such as paramedics and emergency medical technicians (EMTs), may use the measuring device on a person when in a noisy and chaotic environment, such as the middle of a congested street. The emergency responders may also attach the measuring device to a person using an accessory to the measuring device, such as an elastic band or adhesive patch, allowing the emergency responders to move and transport the person while continuously obtaining measurements. Being able to obtain continuous measurements of a person during emergency situations may allow faster and more accurate diagnosis and treatments to be implemented on the person. In another example, emergency responders will be able to use the measuring device to obtain measurements of an injured person and determine the condition and status of the injured person. Knowing the condition and status of the injured person, the emergency responders may better determine if the injured person healthy enough to be transported to a medical facility, such as a hospital, STEMI receiving center, stroke center, etc. The emergency responders may also be able to provide any preliminary medications and treatments to the injured person before and while transporting the injured person to a medical facility. By continuously monitoring the measurements of the injured person, the emergency responders may be able to update preliminary medications and treatments to the injured person, and update their navigational course.

The measuring device may also provide easier monitoring of individuals during or after a mass casualty incident, such as an accident or natural disaster. An emergency responder may be able to attend to multiple individuals at the same time by using a measuring device for each person. By using a measuring device on each person involved in a mass casualty incident, the emergency responder may attend to one person while still obtaining data and a diagnosis of all of the individuals. The emergency responder will also be able to receive immediate feedback from each measuring device allowing for quicker responses and treatment from the emergency responder. This may allow the emergency responder to determine which person of the multiple individuals needs immediate medical treatment, and may save time and resources by allowing the emergency responder to provide accurate treatments to each person. Each measuring device may also send data, alerts and message, of the respective person it is attached to, to medical personnel and rescuers so that accurate treatment may be provided and at a faster response time. The data, alerts and messages may also provide information of any changes that are occurring with the health of the respective person so that treatment may be updated accordingly.

The measuring device may be used on a physiological structure, such as a person, who needs to have their vital signs continuously monitored. The measuring device may continuously obtain vital sign measurements, such as blood pressure, and other data of a person as the measuring device is attached to the person's person. The measuring device may analyze the vital sign measurements and other data that it obtains from the person. The person may program the measuring device to automatically send data to a doctor, or any other individual associated with the measuring device, or manually chose when to send the data. The person may use the measuring device to send all of the obtained and analyzed data to a doctor so the doctor may continuously monitor the person's health. The measuring device may be connected to another device, such as a computer, phone, tablet, etc., to send the data to an individual. The measuring device may be connected to an application or platform, such as telemedicine platforms, where the data may be uploaded for other individuals, such as doctors, nurses, medical practitioners, etc., to access. The measuring device may protect and lock the data by assigning a code or password to the data, and provide such code or password to authorized individuals for access. Providing data to authorized individuals, such as doctors and medical personnel, may help with the study of health issues so that improved and accurate diagnosis and treatment may be provided to individuals. The measuring device may automatically upload data to a cloud database to easily transmit information from patients to their clinical care teams, for research applications, or for the patient's personal information and storage.

The measuring device may send alerts, such as vibrations, sounds, messages, etc., and/or messages to the person attached to the measuring device, when the measuring device determines an issue with the person based on the obtained data. The measuring device may also send alerts and messages to other individuals, such as doctors, family members, friends, etc., when the measuring device determines there is an issue with the person based on the data. The alerts and messages may be sent automatically to authorized individuals. There may be a plurality of alerts that may be sent, where each alert includes a particular message. A message may include data of the person, and may include a recommended diagnosis and treatment for the person (e.g. low blood pressure, go to hospital), based on the data. The measuring device may also provide updates, using alerts and messages, to the person attached to the measuring device, where the updates notify the person of actions the person should take based on the data. Such actions may include visiting a doctor, taking medication, calling for emergency assistance, etc. Alerts and messages sent from the measuring device may help prevent or dissipate medical problems with a person, and help save a person's life when medical assistance is urgently needed.

The measuring device may be used on any individual who needs to be monitored, whether periodically, continuously, or just for a one time occasion. In one example, a person with hypertension may attach and use the measuring device on their person to continuously monitor the person's blood pressure. The measuring device may periodically send data of the person's blood pressure to the person's doctor who has been authorized by the person to access the data. When the measuring device determines, based on the data, that the person's blood pressure is beginning to increase or decrease, the measuring device may send an alert to the person so that the person may implement actions to restabilize their blood pressure. When the measuring device determines, based on the data, that the person is in critical condition and is in need of emergency treatment, the measuring device may send messages to all individuals who have been authorized to receive emergency notifications regarding the person.

In another example, a person with risks for clotting may attach and use the measuring device on their person to continuously monitor the person's vital signs to determine any increased clot burden on the person. The measuring device may also be used on a person who is at risk for deep vein thrombosis (DVT), stroke, pulmonary embolism (PE), apnea, hypotension, hypoxia, and other risks. The measuring device may obtain vital sign data of the person and use the data to assess for microthrombi, blood viscosity, and other metrics. The measuring device may provide feedback, such as recommended diagnosis and treatment, for the patient based on the data assessment. There is no limitation to the type of person or purposes for a person to use the measuring device.

Hospitals and medical personnel may also use the measuring device on patients to monitor their vital signs. Using the measuring device may allow medical personnel to obtain a better understanding of a patient's health and provide better treatment to the patient. In one example, a nurse may use the measuring device on a dialysis patient, who has abnormal blood pressure which can change rapidly during dialysis. The nurse may be able to use the vital sign data of the patient obtained by the measuring device to understand any habits with the patient's vital signs. The nurse may then predict when the patient may have a rapid decrease in blood pressure and adjust the dialysis parameters accordingly to accommodate before the patient experiences symptoms of nausea, dizziness, or syncope. The nurse may also use the measuring device to monitor for any changes in the patient's heart rate, respiratory response, and oxygen saturation (O₂ sat).

Using the measuring device on patients, hospitals and medical personnel may further be able to monitor vital signs of a patient before a procedure is performed on the patient. This may be to ensure that the patient is healthy enough to have the procedure performed on the patient. A patient's health may be determined to be healthy if the measuring device analyzes the data of the patient and determines that the overall health of the patient is above a threshold. The measuring device may also be used on the patient during and after a procedure is performed on the patient to monitor the patient's vital signs to ensure the patient is not experiencing any health issues during and after the procedure. If a health issue is determined by the measuring device, the measuring device may send an alert and message to authorized individuals, such as the patient, medical personnel, family members, etc., to notify them of the issue. This may help with providing accurate treatments to patients and minimalizing any health issues that may occur from performing a procedure on a patient.

The measuring device may be used to determine the health of a person and if the person's health is above a requirement for a particular task or event. In one example, the measuring device may be used on individuals who are traveling long distances, such as a space tourist. In order for a person to be accepted to travel to space as a space tourist, the person may have to have an overall health that is over a threshold. The measuring device may be used to continuously monitor a person for a period of time leading up to space travel, so that administrators may know if the person is healthy enough to be a space tourist upon the day of departure of the space travel. The measuring device may also be able determine if the overall health of a person, based on the data obtained of the person, is above a given threshold to permit the person to join in the space travel. The measuring device may also be used on a person when they are in course of their travels to ensure that any health issues that arise during travel will be found as soon as possible. The measuring device may send an alert and/or message to crew members and/or the ground crew of the flight so that medical attention may be provided to a person experiencing a health issue.

The measuring device may provide an easy means of obtaining vital sign measurements of a person when the person is in various states, such as stressed, relaxed, sleeping, awake, etc. In one example, the measuring device may be used on a person who is easily stressed to monitor the person's vital signs when the person is in various states throughout the day. By continuously monitoring the person's vital signs, the measuring device may accurately diagnosis the person of whether the person has any conditions, such as hypertension. The measuring device may determine that the person only has hypertension when the person is stressed. The measuring device may then send such determination and data of the person to an authorized medical personnel, so the authorized medical personnel may properly diagnose and treat the person. The measuring device may also access the data of vital sign measurements to determine the condition of the person's body, like if the person is overheating, experiencing presyncope, showing signs of illness, fatigue, and the person's overall fitness. This may prevent inaccurate diagnosis and treatments to be provided to a person by the measuring device and medical personnel receiving the data.

The measuring device may be useful to a person in a profession that requires them to be place their body in stressful conditions. In one example, an astronaut may place their body under various stresses, including pressure, atmospheric or temperature changes, when going through launch and reentry of Earth's atmosphere, extravehicular activity (EVA), and periods of spaceflight. The measuring device may enable flight surgeons and other members of an astronaut team to monitor the vital sign measurements of an astronaut during every stage of a space mission to ensure that the astronaut is healthy and not undergoing any issues.

In another example, a soldier may place their body under various stresses when on the battlefield, such as during live ammunition fire. A soldier may include a warfighter, pilot, marine, or any individual serving in a military capacity. The measuring device may monitor vital signs of a soldier and assess injuries that the soldier may have sustained. Based on the data, the measuring device may also send the data, along with alerts and messages, to medical personnel who may attend to the soldier's medical needs. Using the measuring device may help save lives by providing accurate and up-to-date data of a person's health, allowing for accurate diagnosis and treatment to be provided, thus improving assessment and evacuation times. The measuring device may also be used on a soldier during training to determine if the soldier is healthy enough for a particular mission.

The measuring device may provide an easier and more comfortable means in obtaining vital sign measurements of a person who is otherwise unable to do it in the presence of a medical person, such as a doctor. In one example, a person who gets easily stressed and uncomfortable while in the presence of a doctor, or any medical person, may provide difficulty in providing vital sign measurements in the medical person's presence. The measuring device may allow the person to more easily obtain vital sign measurements in a comfortable environment, such as their own home, and may send the data to the medical person or any other authorized individual, or a platform or device. This is particularly relevant to patients with healthcare anxiety or situational/white coat hypertension.

The measuring device may be used by any person who wants to obtain and monitor their vital signs. A person may want to obtain and monitor their vital signs to better understand their body and overall health. A person may want to obtain and monitor their vital signs for a purpose, such as to become fit or meet a goal. The measuring device may be implemented as any device that can be attached to a person who wants or needs to have their vital signs obtained, measured and monitored. In one example, the measuring device may be implemented as a watch that an athlete may wear on their wrist. The athlete may be training for a competition and may need and want to obtain, measure and monitor their vital signs to help improve and adjust their training. Data obtained from the measuring device may be sent to the athlete's trainers so that they may adjust and improve the athlete's training based on the data. In another example, a mountain climber may need to obtain, measure and monitor their vital signs while climbing a mountain. The measuring device may continuously obtain and monitor a mountain climber's vital signs, and use the obtained data to generate acclimatization protocols for the mountain climber. The measuring device may send alerts and messages to the mountain climber to indicate different actions for the mountain climber to take, such as when to ascend, descend, abort, or remain stationary to obtain optimal acclimatization or avoid conditions such as high altitude pulmonary/cerebral edema.

The measuring device may also be helpful on individuals who are in a remote location where medical diagnosis from a medical person is difficult to obtain. Such remote locations may also include locations that do not have access to modern medicine. In one example, the measuring device may be used on a person who is on an expedition and exploring the rain forest of a tropical island. If the measuring device determines that the explorer is having a health issue, based on data obtained from the explorer, then such data may be sent to associated individuals, such as a search and rescue team, who may come to the explorer's aid. The measuring device may also send alerts and messages to any associated individuals, where the alerts and messages may include a recommended diagnosis and treatment determined by the measuring device based on the data. The data, alerts and messages may provide an associated individual with information needed to provide accurate treatment to the explorer, and make decisions such as whether to request a medical evacuation and what type of transportation to request, such as a plane, helicopter, boat, etc. If more than one explorer is on the same expedition, then associated individuals may know how many individuals are in need of medical attention or have had loss of vital signs.

The measuring device may be associated with a company, such as a health provider, insurance provider, etc. A person who is using the measuring device may link a company to the measuring device. The company may receive notifications from the measuring device, where the notifications may include data associated with the vital sign measurements of the person. The company may provide benefits to the person according to data. In one example, an insurance company may determine from the data that the person is within the 90th percentile in health for individuals of similar attributes, such as age, sex, height, etc. The insurance company may provide a discount in health insurance based on the determination. The insurance company may continue to provide a discount in health insurance to the person as the person continues to be within the 90th percentile in health for a particular group of individuals. The discount provided by the insurance company may vary based on the overall health determination of a person from the person's data.

The measuring device may be used on multiple locations on a physiological structure by placing the measuring device against the desired location of the physiological structure, i.e. arm, leg, waist, hip, neck, etc., where vital signs and internal parts of the physiological structure want to be obtained and monitored. The measuring device may also be attached to any location of the physiological structure using an accessory, such as an adhesive or elastic material, that will keep the measuring device in place at the particular location. The measuring device may be attached to the physiological structure for various periods of time to obtain data on the person's vital signs and internal parts of the person at various times of the day, when the person is under various conditions and when the person is performing various tasks.

Following obtaining and monitoring a physiological structure's vital signs, the measuring device may analyze all of the obtained data of the physiological structure and determine a diagnosis for the physiological structure. Non limiting examples of diagnosis that the measuring device may determine are congenital cardiac disorders, pulses and risks for limb ischemia, cardiovascular anomalies such as aortic dissection and vessel occlusion, preeclampsia, sepsis, infection of continuous temp, hypoxia, pneumonia, intubation, complications with pulse oximetry, tachycardia, hypotension, hypertension, internal bleeding, hemorrhage, chronic lung disease, risk of seizures, sleep apnea, postural orthostatic tachycardia syndrome, low blood pressure, low glucose level, deep vein thrombosis (DVT), stroke, pulmonary embolism (PE), superficial thrombophlebitis, blood clotting, tension pneumothorax, supraventricular tachycardia (SVT), idiopathic intermittent atrial fibrillation, angina, myocardial infarction (MI), hyperglycemia, diabetic ketoacidosis (DKT), and other disorders.

Following analyzing a physiological structure's vital signs and internal parts, and diagnosing the physiological structure for any disorders, the measuring device may use all of the data of the physiological structure to predict behaviors of the physiological structure. In one example, a person who is climbing a mountain, i.e. mountain climber, may have a measuring device attached to their waist to monitor their vital signs. After the measuring device has obtained and analyzed data of the mountain climber's vital signs, the measuring device may determine that the mountain climber is at risk of experiencing an illness, injury, disorder, etc. The measuring device may provide an alert and message to the mountain climber to notify the mountain climber of the risks and provide recommendations of how to prevent such illness, injury and/or disorder. Such recommendations may include instructing the climber to stop moving and rest, decrease elevation, contact the base camp medical provider, or take medicine.

In another example, a measuring device attached to a pilot's arm may send an alert to the pilot when the measuring device determines, based on data of the pilot's vital signs, that the pilot is in danger of losing consciousness if the pilot continues to perform dangerous flying maneuvers with the plane.

In another example, a measuring device may be attached to a pregnant woman who is in her second or third trimester. The measuring device may be used to monitor the pregnant woman's vital signs to detect longitudinal change in blood pressure. When increases in blood pressure are detected, the measuring device may send an alert and/or message to a doctor so further analysis may be conducted. Early detection of changes in blood pressure in pregnant women may allow for the prevention and quick diagnosis of eclampsia and preeclampsia, thus allowing for medical attention and treatment to be applied more quickly.

In another example, a measuring device attached to an elderly man to monitor the elderly man's vital signs. The measuring device may determine, based on data of the elderly man's vital signs, that the elderly man will fall (e.g., rapidly progressive hypotension and/or tachycardia plus or minus accelerometry). The measuring device may send an alert and/or message to the elderly man to notify the elderly man that he is about to fall so that he may sit or lie down prior to collapsing. The measuring device may also include an accelerometer to detect when the elderly man has fallen.

The measuring device may also send an alert and/or message to other individuals, such as family members, doctors, emergency responders, etc., who have been authorized and listed in the measuring device to send alerts and messages to. The measuring device may be able to predict a variety of illnesses, disorders, injuries, etc., that a physiological structure may experience based on data of the physiological structure's vital signs and internal parts. The measuring device may also use all of the data of the physiological structure that it has obtained to determine that particular illnesses, disorders, injuries, conditions, etc., are present in the physiological structure. In this way, the measuring device may both predict future and determine present attributes and conditions relating to the vital signs, internal parts, and overall health of a physiological structure.

In an example, a monitoring device coupled to a subject may be used to continuously measure blood pressure of the subject and to determine a stress level of the subject at least in part from the blood pressure of the subject. In some cases, the monitoring device may provide a real-time or continuously updated stress level for the subject. In some cases, the monitoring device may provide an alert to the subject or to an external party if the subject's stress level reaches a stress level alert threshold. The stress level alert threshold may be based at least in part on historical blood pressure data of the subject, health status of the subject, medical records of the subject, or any combination thereof. In some cases, the device is a portable or wearable device configured to continuously monitor blood pressure of a patient over a long-duration or a period of high-stress or exertion. In some cases, the device is configured to measure and monitor BP for pilots, truck drivers, racecar drivers, or astronauts. In some cases, the subject is subjected to a physical stressor and the stress level of the subject is usable to determine a time point at which continued exposure to the physical stressor may cause adverse health outcomes for the subject. The stress level of the subject may be used to determine a time point at which the physical stressor may be removed to avoid adverse health outcomes for the subject. In some cases, the device is wearable. In some cases, the device is a wrist band, arm band, wearable patch, or integrated into clothing. In some cases, a portion of the device is configured to be mounted on a structure of platform used by or near the subject. The device can be configured to sample BP at high temporal resolution (e.g., continuously, beat-to-beat, or minute-level). In some cases, the device is configured to further recording posture, motion, and other physiologic signals (PPG, accelerometry, audio-derived pulse features) to filter out motion artifact. In some cases, the device can timestamp each reading. In some cases, each reading may comprise a tag with quality metrics (for example motion index, sensor SNR) or contextual markers (for example mission phase, driving shift, or exertion period), to help identify real physiologic changes from noise. In some cases, the device can run onboard processing to compute variability metrics and trigger configurable alerts for extreme events (for example large SBP swings, sustained hypertension, or dangerous drops on standing). In some cases, the device is configured to securely stream or batch-export annotated time-series and reports to ground teams, medical support, or fleet health systems for real-time or post-event review. In some cases, the device can operate with low power and resilient connectivity. In some cases, the device supports calibration routines and multi-sensor fusion to maintain measurement accuracy across prolonged use in different environments.

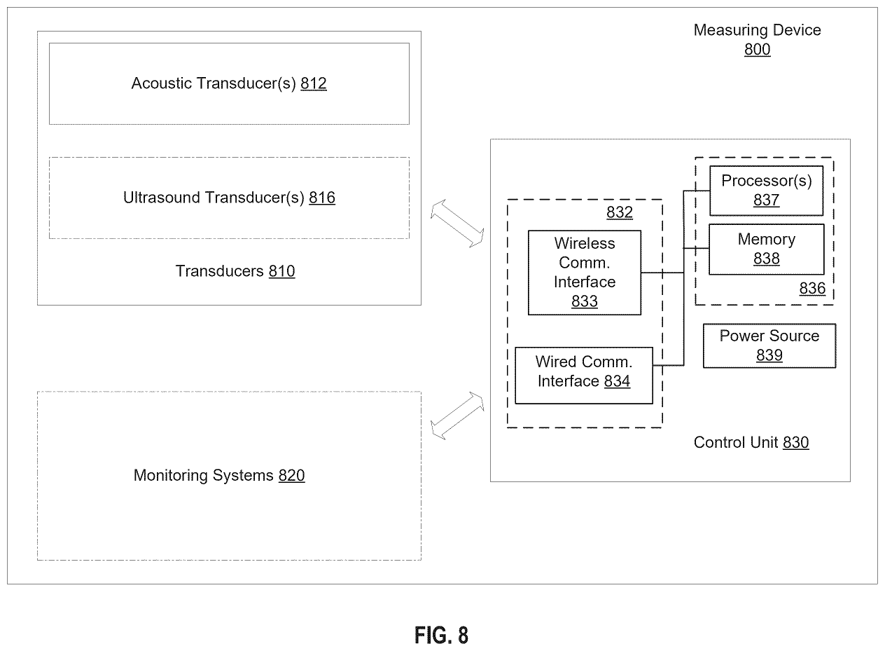

FIG. 8 illustrates an example measuring device 800, in accordance with various embodiments of the presently disclosed technology.

As depicted, measuring device 800 comprises a control unit 830 and transducers 810. In some embodiments, measuring device 800 may further comprise a monitoring system 820 (described in greater detail below).

As depicted, transducers 810 may comprise acoustic transducer(s) 812. Acoustic transducer(s) 812 may comprise one or more acoustic transducers. As used herein, an acoustic transducer may refer to a device that: (a) transmits acoustic energy (e.g., a speaker); (b) acquires/receives acoustic energy (e.g., a microphone); or (c) transmits and receives acoustic energy (e.g., a transceiver than includes both acoustic transmitter and acoustic receiver components). An acoustic transducer that transmits acoustic energy may convert received electrical signals into the acoustic energy/acoustic signals it transmits. An acoustic transducer that acquires/receives acoustic energy may convert acquired/received acoustic energy into electrical signals. An acoustic transducer that operates in a “transmit only” mode may convert impedance measured from the transducer into electrical signals. As used herein, an acoustic transducer that transmits acoustic energy but does not receive/acquire acoustic energy (e.g., a speaker) may be referred to as a non-receiver acoustic transducer, an acoustic transmitter, an ultrasound transmitter, or similar. In general, non-receiver acoustic transducers (e.g., speakers) are less expensive and consume less power than acoustic transducers that both transmit and receive acoustic energy (e.g., acoustic transceivers).

Certain embodiments can reduce expense and power consumption by using non-receiver acoustic transducers in measuring devices of the presently disclosed technology. For example, acoustic transducer(s) 812 may comprise one or more non-receiver acoustic transducers. Such a non-receiver acoustic transducer can be used to transmit acoustic energy of different frequencies towards a blood vessel in order to probe/determine the blood vessel's resonant frequency. In general, the blood vessel will absorb some of the acoustic energy, and reflect some of the acoustic energy back. As embodiments of the presently disclosed technology are designed in appreciation of, when the blood vessel is hit with acoustic energy at a resonant frequency of a wall of the blood vessel (i.e., a blood vessel wall), a significantly greater amount of the acoustic energy will be absorbed by the blood vessel wall, and less acoustic energy will be reflected back at the non-receiver acoustic transducer. For example, embodiments can use the non-receiver acoustic transducer to transmit acoustic energy at a first frequency and then transmit acoustic energy at a second frequency. Here, the second frequency may correspond with the resonant frequency of the blood vessel wall. Accordingly, responsive to transmission of acoustic energy at the second frequency, the blood vessel wall may reflect significantly less acoustic energy back to the non-receiver acoustic transducer.

However, lacking an acoustic energy receiving component, the non-receiver acoustic transducer would generally not be able to detect the change in acoustic energy that is reflected back to the non-receiver acoustic transducer when the blood vessel is hit with acoustic energy at its resonant frequency (i.e., the second frequency). Embodiments are intelligently designed to overcome this technical challenge by measuring an electrical property of the non-receiver acoustic transducer (e.g., current, voltage, power, resistance, impedance, etc.) as a proxy for measuring the acoustic energy that is reflected back to the non-receiver acoustic transducer. This is based on an intelligent insight that electrical properties of the non-receiver acoustic transducer may be impacted as a function of the magnitude of acoustic energy propagating towards the non-receiver acoustic transducer in a direction that opposes the direction the non-receiver acoustic transducer transmits audio energy (akin to pushing a door into the wind). For example, the amount of electrical power the non-receiver acoustic transducer requires to transmit acoustic energy may increase when other acoustic energy is propagating back towards the non-receiver acoustic transducer in an opposing direction. In other words, the non-receiver acoustic transducer may require less electrical power to transmit acoustic energy when a lesser magnitude of acoustic energy is reflected back to the non-receiver acoustic transducer from the blood vessel wall. Accordingly, embodiments can determine a resonant frequency of the blood vessel wall as the transmission frequency at which an amount of power required to transmit is reduced/minimized.

In other words, embodiments can use the non-receiver acoustic transducer to transmit acoustic energy at a first frequency and then transmit acoustic energy at a second frequency. In this embodiment, the second frequency may correspond with the resonant frequency of the blood vessel wall and absorb more energy when transmitted. By measuring the impedance of the non-receiver acoustic transducer, the resonant frequency of the blood vessel wall can be determined without the need for a receiver. Namely, when a non-receiver acoustic transducer emits a non-resonant frequency towards a blood vessel, the impedance measured from the non-receiver acoustic transducer may be higher, and when a non-receiver acoustic transducer emits a resonant frequency towards a blood vessel, the impedance measured from the non-receiver acoustic transducer may be lower. This process may be repeated as many times as necessary (e.g., nth frequency to nth+1 frequency) to discover a local impedance minima, which may correlate to a resonant frequency of the blood vessel wall.

However, it should be understood that in other embodiments acoustic transducer(s) 812 may comprise acoustic energy transceivers that can both transmit and receive acoustic energy. In these embodiments, measuring device 800 can determine the resonant frequency of the blood vessel wall by analyzing acoustic energy that is received by acoustic transducer(s) 812 after being reflected back from the blood vessel.

As depicted, in certain embodiments transducers 810 may also comprise ultrasound transducer(s) 816. Ultrasound transducer(s) 816 may comprise one or more ultrasound transducers capable of transmitting and receiving ultrasound energy (i.e., acoustic energy involving ultrasound signals). While ultrasound transducer(s) 816 may be more expensive and consume more power than non-ultrasound acoustic transducers, ultrasound transducer(s) 816 can be useful for imagining the blood vessel. Accordingly, measuring device 800 can utilize the imagining capabilities of ultrasound energy to determine wall thickness of the blood vessel and blood vessel radius of the blood vessel. As alluded to above, measuring device 800 can use these determined parameters, along with determined resonant frequency of the blood vessel wall, to generate a blood pressure measurement. While wall thickness and blood vessel radius generally remain relatively consistent across individuals, precise/individualized measurements of these parameters can lead to more accurate blood pressure measurements. Accordingly, leveraging ultrasound transducer(s) 816 to determine wall thickness of the blood vessel and blood vessel radius of the blood vessel, measuring device 800 can improve blood pressure measurement accuracy. However, in other embodiments (e.g., where ultrasound transducer(s) 816 are not included in transducers 810 (e.g., to reduce cost and power consumption), measuring device 800 can rely on estimated values for these parameters acquired from applicable medical databases/medical literature. In still further embodiments, measuring device 800 can utilize ultrasound transducer(s) 816 to initially measure wall thickness and blood vessel radius of the blood vessel, and then rely on lower power-consuming non-ultrasound transducers for continued monitoring of blood vessel wall resonant frequency as embodiments do not require ultrasound transducers for such monitoring.

As depicted, measuring device 800 also comprises a control unit 830. Components within control unit 830 can communicate via a data bus, and/or other suitable communication interfaces.

Communication circuit 832 may comprise at least one of a wireless communication interface 833 (e.g., a transceiver with an antenna) and a wired communication interface 834 (e.g., an I/O interface with an associated hardwired data port). Control unit 830 can utilize communication circuit 832 to communicate with transducers 810 and monitoring systems 820. Control unit 830 can also utilize communication circuit 832 to communicate with devices remote from measuring device 800. For example, in certain implementations monitoring systems 820 may be located remotely from measuring device 800.

Wireless communication interface 833 may include a transceiver (i.e., a receiver and transmitter) to allow wireless communications via various communication protocols such as, WiFi, Zigbee, Bluetooth, near field communications, etc. As alluded to above, wireless communication interface 833 may comprise an antenna coupled to the transceiver to send and receive radio signals wirelessly. These radio signals can include information sent to and from transducers 810 and monitoring systems 820. These radio signals can also include radio signals sent to and from devices remote from measuring device 800.

Wired communication interface 834 can include a receiver and a transmitter for hardwired communications with other components of measuring device 800 (e.g., transducers 810 and monitoring systems 820). For example, wired communication interface 834 can provide a hardwired interface to other components, including transducers 810 and monitoring systems 820. Wired communication interface 834 can communicate with these components using Ethernet or any number of other wired communication protocols. In various examples, wired communication interface 834 can communicate with devices remote from measuring device 800.

As depicted, determination circuit 836 includes processor(s) 837 and memory 838. Processor(s) 837 can include one or more processing resources, such as GPUs, CPUs, microprocessors, etc.

Memory 838 may comprise one or more modules of various forms of memory/data storage (e.g., flash, RAM, etc.) for storing the various data, parameters, and operational instructions utilized by processor(s) 837, as well as any other suitable information.

While the specific example of FIG. 8 is illustrated using processor and memory circuitry, determination circuit 836 can be implemented utilizing any form of circuitry including, for example, hardware, software, or a combination thereof. As a further example, one or more processors, controllers, ASICs, PLAS, PALs, CPLDs, FPGAs, logical components, software routines or other mechanisms may be used to implement control unit 830.

Power source 839 may comprise any type of suitable power source. For instance, power supply 839 can include one or more batteries (e.g., rechargeable or primary batteries comprising Li-ion, Li-Polymer, NiMH, NiCd, NiZn, NiH2, etc.), a power connector (e.g., to connect to supplied power), and an energy harvester (e.g., solar cells, piezoelectric system, etc.).

As alluded to above, in certain embodiments measuring device 800 may include monitoring systems 820. In other embodiments, monitoring systems 820 may comprise a remotely located, separate system. Monitoring systems 820 can display information to users related to monitored blood pressure. For example, control unit 830 can generate a blood pressure measurement, and then send a notification to monitoring systems 820 that contains the generated blood pressure measurement. Monitoring systems 820 may comprise a graphical user interface (GUI) that displays the notification to a user.

In some implementations, measuring device 800 may comprise a wearable cuff dimensioned to be worn around a person's limb (e.g., an armband). The blood vessel may be located within the person's limb. Here, transducers 810 may be mechanically attached to the wearable cuff such that they contact the person's tissue.

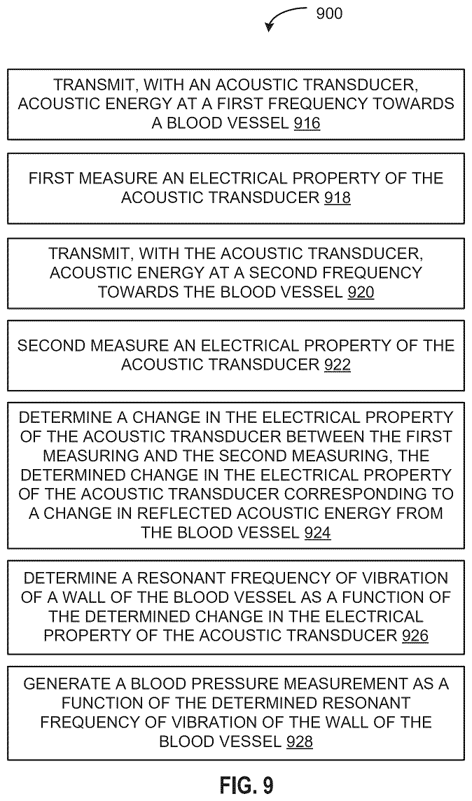

FIG. 9 illustrates an example methodology 900 for generating a blood pressure measurement as a function of a determined resonant frequency of vibration of a wall of a blood vessel.

As depicted, operation 916 may comprise transmitting, with an acoustic transducer, acoustic energy at a first frequency towards a blood vessel (e.g., an artery, a vein, a capillary, etc.). In certain embodiments, the acoustic transducer may comprise a non-receiver acoustic transducer. In various implementations, the blood vessel may be in a physiological structure and transmitting acoustic energy towards the blood vessel can comprise transmitting acoustic energy towards the blood vessel through the physiological structure.

Operation 918 may comprise first measuring an electrical property of the acoustic transducer. As alluded to above, the electrical property may comprise at least one of: current; power; voltage; resistance; and impedance. Here, first measuring the electrical property of the acoustic transducer may be responsive to transmission of the acoustic energy at the first frequency.

Operation 920 may comprise transmitting, with the acoustic transducer, acoustic energy at a second frequency towards the blood vessel. In certain examples, the second frequency may correspond with the resonant frequency of vibration of the wall of the blood vessel.

Operation 922 may comprise second measuring the electrical property of the acoustic transducer. Second measuring the electrical property of the acoustic transducer may be responsive to transmission of the acoustic energy at the second frequency.

Operation 924 may comprise determining a change in the electrical property of the acoustic transducer between the first measuring and the second measuring, the determined change in the electrical property of the acoustic transducer corresponding to a change in reflected acoustic energy from the blood vessel.

Operation 926 may comprise determining a resonant frequency of vibration of a wall of the blood vessel as a function of the determined change in the electrical property of the acoustic transducer.

Operation 928 may comprise generating a blood pressure measurement as a function of the determined resonant frequency of vibration of the wall of the blood vessel. In certain implementations, generating the blood pressure measurement may comprise generating the blood pressure measurement as a function of: the determined resonant frequency of vibration of the wall of the blood vessel; estimated wall thickness of the blood vessel; and estimated vessel radius of the blood vessel.

In certain implementations, methodology 900 may further comprise sending, to a monitoring system, a notification containing the generated blood pressure measurement.

In some implementations, methodology 900 may further comprise: (a) transmitting, with the acoustic transducer, acoustic energy at a third frequency towards the blood vessel; (b) third measuring the electrical property of the acoustic transducer; and (c) determining a second change in the electrical property of the acoustic transducer between the second measuring and the third measuring, the determined second change in the electrical property of the transducer corresponding to a second change in reflected acoustic energy from the blood vessel. Here, determining the resonant frequency of vibration of the wall of the blood vessel may comprise determining the resonant frequency of vibration of the wall of the blood vessel as a function of the first and second determined changes in reflected acoustic energy from the blood vessel. Use of the third frequency here may result in improved accuracy/precision for determining the resonant frequency of vibration of the wall of the blood vessel.

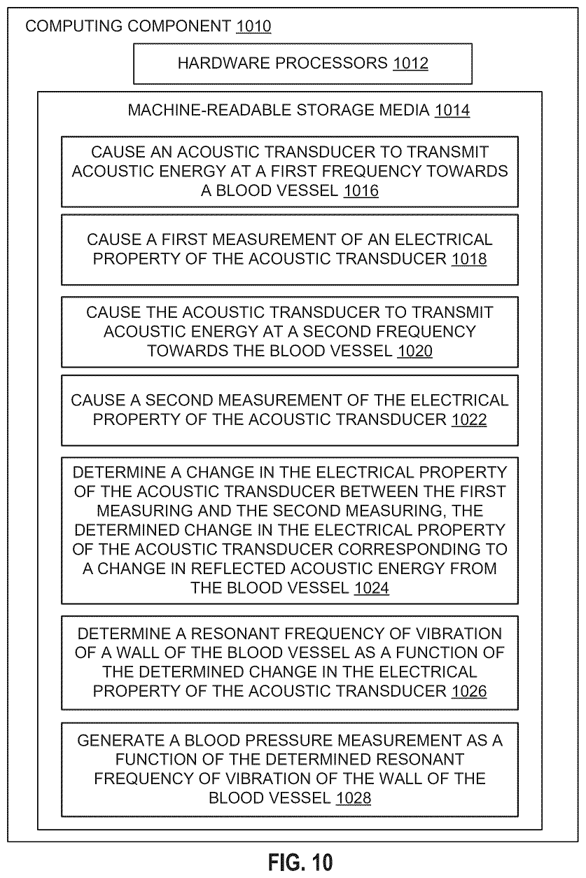

FIG. 10 is a companion figure to FIG. 9 that depicts how the methodology 900 may be implemented using a computing component 1010. Here, computing component 1010 may be implemented on control unit 830 of FIG. 8. While the instructions of FIG. 10 will not be described again for brevity, other components of computing component 1010 are described here.

Computing component 1010 may be, for example, a server computer, a controller, or any other similar computing component capable of processing data. In the example implementation of FIG. 10, the computing component 1010 includes a hardware processor 1012, and machine-readable storage medium for 1014.

Hardware processor 1012 may be one or more central processing units (CPUs), semiconductor-based microprocessors, and/or other hardware devices suitable for retrieval and execution of instructions stored in machine-readable storage medium 1014. Hardware processor 1012 may fetch, decode, and execute instructions, such as instructions 1016-1028, to control processes or operations. As an alternative or in addition to retrieving and executing instructions, hardware processor 1012 may include one or more electronic circuits that include electronic components for performing the functionality of one or more instructions, such as a field programmable gate array (FPGA), application specific integrated circuit (ASIC), or other electronic circuits.

A machine-readable storage medium, such as machine-readable storage medium 1014, may be any electronic, magnetic, optical, or other physical storage device that contains or stores executable instructions. Thus, machine-readable storage medium 1014 may be, for example, Random Access Memory (RAM), non-volatile RAM (NVRAM), an Electrically Erasable Programmable Read-Only Memory (EEPROM), a storage device, an optical disc, and the like. In some examples, machine-readable storage medium 1014 may be a non-transitory storage medium, where the term “non-transitory” does not encompass transitory propagating indicators. As described in detail below, machine-readable storage medium 1014 may be encoded with executable instructions, for example, instructions 1016-1028.

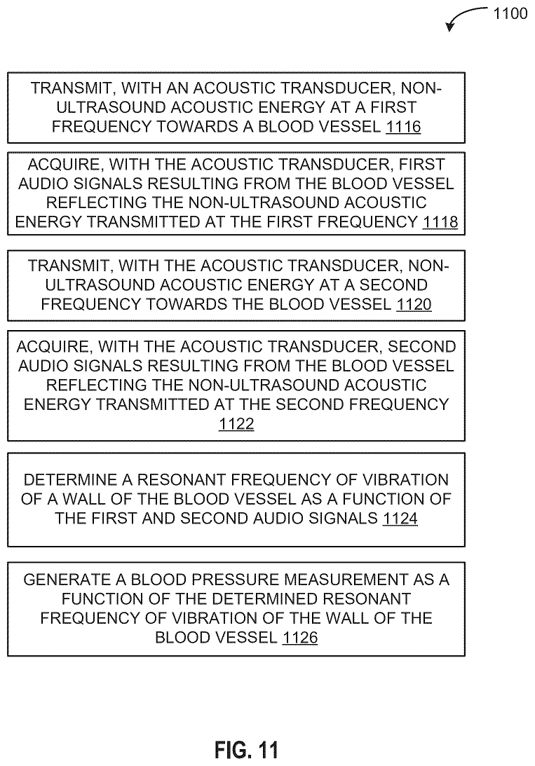

FIG. 11 illustrates an example methodology 1100 for generating a blood pressure measurement as a function of a determined resonant frequency of vibration of a wall of a blood vessel.

Operation 1116 may comprise transmitting, with an acoustic transducer, non-ultrasound acoustic energy at a first frequency towards a blood vessel. As alluded to above, generating non-ultrasound acoustic energy instead of ultrasound acoustic energy can reduce expense and power consumption.

Operation 1118 may comprise acquiring, with the acoustic transducer, first audio signals resulting from the blood vessel reflecting the non-ultrasound acoustic energy transmitted at the first frequency.

Operation 1120 may comprise transmitting, with the acoustic transducer, non-ultrasound acoustic energy at a second frequency towards the blood vessel.

Operation 1122 may comprise acquiring, with the acoustic transducer, second audio signals resulting from the blood vessel reflecting the non-ultrasound acoustic energy transmitted at the second frequency.

Operation 1124 may comprise determining a resonant frequency of vibration of a wall of the blood vessel as a function of the first and second audio signals.

Operation 1126 may comprise generating a blood pressure measurement as a function of the determined resonant frequency of vibration of the wall of the blood vessel. Here, generating the blood pressure measurement may comprise generating the blood pressure measurement as a function of: the determined resonant frequency of vibration of the wall of the blood vessel; estimated wall thickness of the blood vessel; and estimated vessel radius of the blood vessel. As alluded to above, wall thickness and vessel radius may be estimated here to account for lack of imagining by ultrasound for determining these parameters.

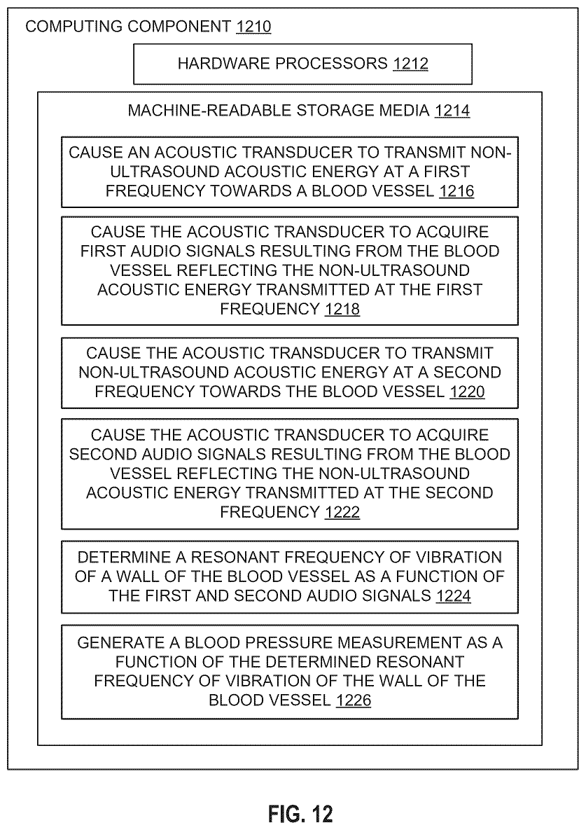

FIG. 12 is a companion figure to FIG. 11 that illustrates how methodology 1100 may be performed by a computing component 1210. Like computing component 1010, computing component 1210 may be implemented on control unit 830 of FIG. 8. The instructions of FIG. 12 (i.e., instructions 1214-1226) will not be described again for brevity. Hardware processor 1212 and machine-readable storage media 1214 may be the same/similar as hardware processor 1012 and machine-readable storage media 1014 of FIG. 10.

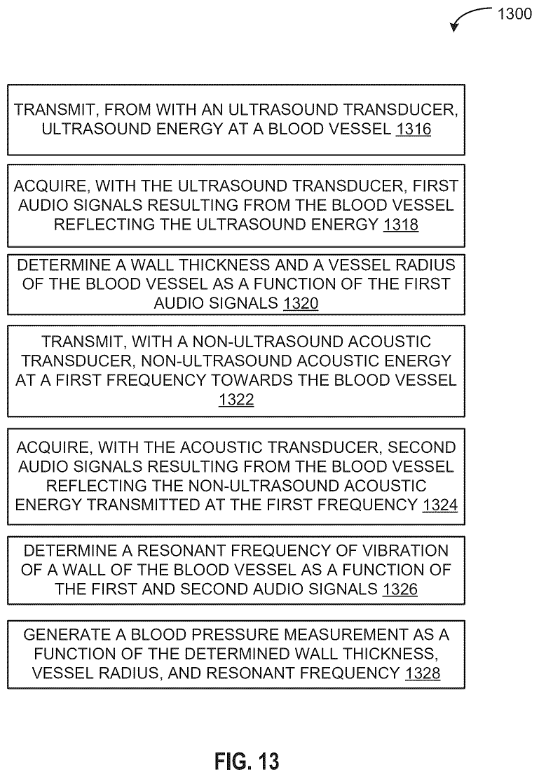

FIG. 13 illustrates an example methodology 1300 for generating a blood pressure measurement as a function of a determined resonant frequency of vibration of a wall of a blood vessel.

Operation 1316 may comprise transmitting, from with an ultrasound transducer, ultrasound energy at a blood vessel.

Operation 1318 may comprise acquiring, with the ultrasound transducer, first audio signals resulting from the blood vessel reflecting the ultrasound energy.

Operation 1320 may comprise determining a wall thickness and a vessel radius of the blood vessel as a function of the first audio signals.

Operation 1322 may comprise transmitting, with a non-ultrasound acoustic transducer, non-ultrasound acoustic energy at a first frequency towards the blood vessel.

Operation 1324 may comprise acquiring, with the acoustic transducer, second audio signals resulting from the blood vessel reflecting the non-ultrasound acoustic energy transmitted at the first frequency.

Operation 1326 may comprise determining a resonant frequency of vibration of a wall of the blood vessel as a function of the first and second audio signals.

Operation 1328 may comprise generating a blood pressure measurement as a function of the determined wall thickness, vessel radius, and resonant frequency.

As alluded to above, embodiments can conserve power consumption by only using an ultrasound transducer to initially determine blood vessel wall thickness and blood vessel radius (which generally cannot be determined using non-ultrasound transducers), and then only using non-ultrasound transducers for continued monitoring/determination of resonant frequency of vibration of the wall of the blood vessel.

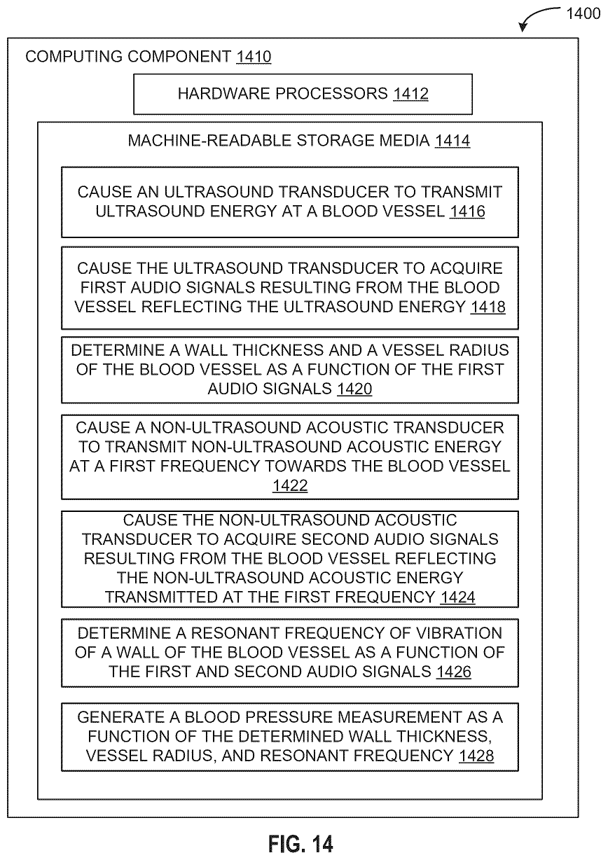

FIG. 14 is a companion figure to FIG. 13 that illustrates how methodology 1300 may be performed by a computing component 1410. Like computing component 1010, computing component 1410 may be implemented on control unit 830 of FIG. 8. The instructions of FIG. 14 (i.e., instructions 1414-1428) will not be described again for brevity. Hardware processor 1412 and machine-readable storage media 1414 may be the same/similar as hardware processor 1012 and machine-readable storage media 1014 of FIG. 10.

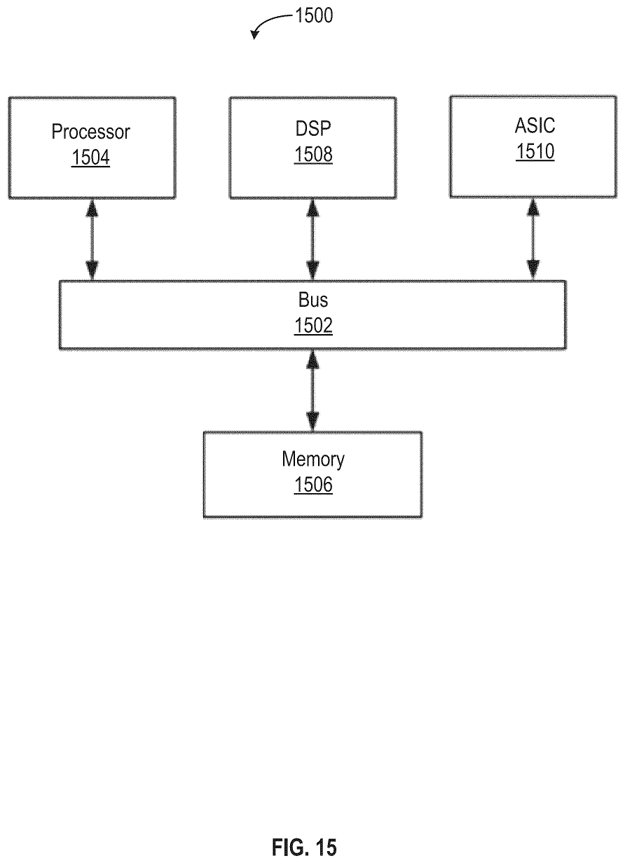

FIG. 15 illustrates a chip set 1500 in which embodiments of the disclosure may be implemented. Chip set 1500 can include, for instance, processor and memory components incorporated in one or more physical packages. By way of example, a physical package includes an arrangement of one or more materials, components, and/or wires on a structural assembly (e.g., a baseboard) to provide one or more characteristics such as physical strength, conservation of size, and/or limitation of electrical interaction.

In one embodiment, chip set 1500 includes a communication mechanism such as a bus 1502 for passing information among the components of the chip set 1500. A processor 1504 has connectivity to bus 1502 to execute instructions and process information stored in a memory 1506. Processor 1504 includes one or more processing cores with each core configured to perform independently. A multi-core processor enables multiprocessing within a single physical package. Examples of a multi-core processor include two, four, eight, or greater numbers of processing cores. Alternatively or in addition, processor 1504 includes one or more microprocessors configured in tandem via bus 1502 to enable independent execution of instructions, pipelining, and multithreading. Processor 1504 may also be accompanied with one or more specialized components to perform certain processing functions and tasks such as one or more digital signal processors (DSP) 1508, and/or one or more application-specific integrated circuits (ASIC) 1510. DSP 1508 can typically be configured to process real-world signals (e.g., sound) in real time independently of processor 1504. Similarly, ASIC 1510 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

Processor 1504 and accompanying components have connectivity to the memory 1506 via bus 1502. Memory 1506 includes both dynamic memory (e.g., RAM) and static memory (e.g., ROM) for storing executable instructions that, when executed by processor 1504, DSP 1508, and/or ASIC 1510, perform the process of example embodiments as described herein. Memory 1506 also stores the data associated with or generated by the execution of the process.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

Each of the processes, methods, models, and algorithms described in the preceding sections may be embodied in, and fully or partially automated by, code components executed by one or more computer systems or computer processors comprising computer hardware. The processes, models, and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. Additionally, unless the context dictates otherwise, the methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computers processors, not only residing within a single machine, but deployed across a number of machines.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

1. A blood vessel locating device, comprising:
a plurality of ultrasound sensors configured to capture tomographical information of a physiological structure;
an ultrasound coupling medium disposed on an ultrasound sensor of the plurality of ultrasound sensors; and
a processing device configured to:
apply beamforming techniques to the plurality of ultrasound sensors;
capture, using the plurality of ultrasound sensors, bodily data of overlapping volumes of the physiological structure;
process the bodily data to generate a vessel location model; and
locate a blood vessel in the physiological structure from the vessel location model.

2. The blood vessel locating device of claim 1, wherein the ultrasound sensor of the plurality of ultrasound sensors comprises a transducer, and wherein the transducer comprises a low pixel-count array.

3-6. (canceled)

7. The blood vessel locating device of claim 1, wherein the processing device is further configured to: (i) determine a plurality of blood pressure measurements of a subject from the blood vessel located in the physiological structure and (ii) using at least in part the plurality of blood pressure measurements and medical records of the subject, determine a health status of the subject.

8-10. (canceled)

11. The blood vessel locating device of claim 1, wherein the processing device is configured to capture bodily data of overlapping volumes of the physiological structure by:
acquiring a velocity of blood moving through the blood vessel;
acquiring a resonance response of the blood vessel to an audio-frequency sound stimulus applied to the blood vessel; and
capturing image data of the blood vessel and surrounding structures near the blood vessel that are detectable by the plurality of ultrasound sensors, wherein the surrounding structures comprise:
internal parts, wherein the internal parts comprise organs, muscles, bones, tissues, and tendons;
pathologies, wherein the pathologies comprise fractures, abscesses, tumors, cellulitis, and stones; and
properties associated with the blood vessel, internal parts, and pathologies.

12. (canceled)

13. The blood vessel locating device of claim 11, wherein the processing device is further configured to:
compare the velocity of the blood moving through the blood vessel to a first threshold and the resonance response of the blood vessel to a second threshold; and
track the blood vessel upon determining that the velocity of the blood moving through the blood vessel is greater than the first threshold and the resonance response of the blood vessel is greater than the second threshold.

14. The blood vessel locating device of claim 1, wherein the processing device is configured to process the bodily data to generate the vessel location model by:
generating the vessel location model using the bodily data; and
acquiring, using the bodily data, arterial stiffness, wall thickness, and arterial diameter of the blood vessel to apply to the vessel location model.

15. The blood vessel locating device of claim 11, wherein the processing device is further configured to:
generate an image of the blood vessel and the surrounding structures based on the image data.

16. (canceled)

17. (canceled)

18. A method, comprising:
applying beamforming techniques to a plurality of ultrasound sensors, wherein an ultrasound sensor the plurality of ultrasound sensors comprises an ultrasound coupling medium disposed thereon;
capturing, using the plurality of ultrasound sensors, bodily data of overlapping volumes of a physiological structure;
processing the bodily data to generate a vessel location model; and
locating a blood vessel in the physiological structure from the vessel location model.

19. (canceled)

20. The method of claim 18, wherein the ultrasound sensor of the plurality of ultrasound sensors comprises a transducer, and wherein the transducer uses phased-array technology.

21. The method of claim 18, further comprising (i) determining a plurality of blood pressure measurements of a subject from the blood vessel located in the physiological structure and (ii) from the plurality of blood pressure measurements and medical records of the subject, determining a health status of the subject.

22. The method of claim 21, further comprising (I) generating an alert threshold based at least in part on the health status of the subject and (II) providing an alert to the subject or an external party upon determining blood pressure measurements of the plurality of blood pressure measurements are greater than or less than the alert threshold.

23. The method of claim 21, further comprising (I) monitoring a variability in the plurality of blood pressure measurements over time and (II) providing the variability in the plurality of blood pressure measurements over time to electronic medical records of the subject.

24. The method of claim 21, further comprising (I) determining a stress level of the subject from the plurality of blood pressure measurements and (II) providing an alert to the subject or an external party upon the stress level of the subject reaching a threshold stress level.

25. (canceled)

26. The method of claim 18, wherein the capturing bodily data of overlapping volumes of the physiological structure comprises:
acquiring a velocity of blood moving through the blood vessel;
acquiring a resonance response of the blood vessel to an audio-frequency sound stimulus applied to the blood vessel; and
capturing image data of the blood vessel and surrounding structures near the blood vessel that are detectable by the plurality of ultrasound sensors, wherein the surrounding structures comprises:
internal parts, wherein the internal parts comprise organs, muscles, bones, tissues, and tendons;
pathologies, wherein the pathologies comprise fractures, abscesses, tumors, cellulitis, and stones; and
properties associated with the blood vessel, internal parts, and pathologies.

27. The method of claim 24, further comprising using the ultrasound sensor of the plurality of ultrasound sensors to apply the audio-frequency sound stimulus to the blood vessel.

28. The method of claim 26, further comprising:
comparing the velocity of blood moving through the blood vessel to a first threshold and the resonance response of the blood vessel to a second threshold; and
tracking the blood vessel upon determining that the velocity of the blood moving through the blood vessel is greater than the first threshold and the resonance response of the blood vessel is greater than the second threshold.

29. A blood vessel monitoring device, comprising:
a plurality of non-receiving transducers configured to transmit energy towards a blood vessel;
a coupling medium disposed on a non-receiving transducer of the plurality of non-receiving transducers; and
a processing device configured to:
direct energy at a first frequency towards the blood vessel;
capture a first set of data generated in response to the energy at the first frequency, wherein the first set of data corresponds to a first impedance measurement of the non-receiving transducer of the plurality of non-receiving transducers;
direct energy at a second frequency towards the blood vessel;
capture a second set of data generated in response to the energy at the second frequency, wherein the second set of data corresponds to a second impedance measurement of the non-receiving transducer of the plurality of non-receiving transducers; and
compare the first set of data and the second set of data to generate location data or attribute data of the blood vessel.

30-32. (canceled)

33. The device of claim 29, wherein the first frequency and the second frequency are different frequencies.

34. The device of claim 29, wherein the processing device is further configured to: (i) direct additional frequencies towards the blood vessel; (ii) capture additional sets of data corresponding to additional impedance measurements, and (iii) determine a lowest impedance measurement of the non-receiving transducer by comparing nth sets of data to nth+1 sets of data, wherein the lowest impedance measurement is indicative of a resonant frequency of the blood vessel wall.

35. The blood vessel monitoring device of claim 29, wherein the processing device is further configured to: (i) determine a plurality of blood pressure measurements of a subject from the attribute data of the blood vessel and (ii) using the plurality of blood pressure measurements and medical records of the subject, determine a health status of the subject.

36-38. (canceled)
